(19) United States
(12) Patent Application Publication
(10) Pub. No.: US 2016/0270902 A1
(43) Pub. Date: Sep. 22, 2016

(54) DEVICE FOR FIXATION OF A FLEXIBLE ELEMENT, PARTICULARLY A NATURAL OR SYNTHETICAL LIGAMENT OR TENDON, TO A BONE

(71) Applicant: UNIVERSITÄT ZÜRICH, Zürich (CH)

(72) Inventors: Jess G. SNEDEKER, Zurich (CH); Xiang LI, Zurich (CH); Hans Rudolf SOMMER, Monchaltorf (CH)

(73) Assignee: UNIVERSITAT ZURICH, Zurich (CH)

(21) Appl. No.: 14/442,404

(22) PCT Filed: Nov. 12, 2013
(86) PCT No.: PCT/EP2013/073759

§ 371 Date: May 13, 2015

(30) Foreign Application Priority Data
Nov. 13, 2012 (EP) 12192333.8

(51) Int. Cl. A61F 2/08

(52) U.S. Cl. CPC A61F 2/0805; A61F 2002/0835; A61F 2002/0852; A61F 2002/0888

(57) ABSTRACT

The invention relates to a device (1) for fixing a flexible element (10), particularly in the form of an artificial or natural ligament or a tendon, to a bone (20), comprising: an insert (100) being designed to hold said flexible element (10), and an anchor (200), wherein the insert (100) is designed to be inserted into said anchor (200), and wherein the anchor (200) is designed to be inserted into a bore hole (2) of said bone (20) together with said insert (100) inserted into the anchor (200) to fix the flexible element (10) to the bone (20).

The invention relates to a device for fixing a flexible element, particularly in the form of an artificial or natural ligament or an artificial or natural tendon, to a bone, preferably to a human bone.

Due to anatomical locations, such flexible elements, like the anterior cruciate ligament (ACL) for instance, are subjected to bear tremendous forces during sports and other daily activities. The ACL rupture is regarded as the most frequent and severe ligament injury [1]. It has been estimated that there are around 250,000 (or 1 in 3,000 in the general population) patients per year diagnosed with ACL disruption in the United States, with approximately 75,000 performed surgical reconstructions annually [2-5]. In Switzerland, there are around 5,000 ACL reconstructions each year. Although many surgical options for ACL reconstruction, including autografts, allografts, xenografts, or synthetic grafts, have been practiced for the restoration of knee joint stability, several unavoidable drawbacks exist, such as donor site morbidity [6, 7], disease transmission [8], immune response [9, 10], ligament laxity [11], mechanical mismatch, and so on [12, 13]. Therefore, more optimal reconstructive techniques for ACL repair are required and should be developed. The rapid development of tissue engineering technique offers a promising approach of regenerating functional tissues to treat ACL injuries [5, 14-19].

It is regarded that biomaterial scaffolds are a key factor in tissue engineering. An ideal ACL replacement scaffold should be biodegradable, biocompatible, with suitable porosity for the cell ingrowth, and sufficient mechanical stability [12, 14]. Silkworm silk fibroin, a natural biopolymer usable after removal of the hyper-allergenic sericin component from raw silk [20, 21], has been used as clinical suture material for centuries [22]. Silk fibroin provides an excellent combination of outstanding and customizable mechanical properties (up to 4.8 GPa), remarkable toughness and elasticity (up to 35%), and environmental stability [15, 23, 24]. As a structural template, silk fibroin has been shown to bear equivalence to collagen in supporting cell attachment, inducing appropriate morphology and cell growth [25, 26], with a degradation rate that involves a gradual loss of tensile strength over 1 year in vivo [5, 23]. Thus, because of good biocompatibility, biomechanical properties, and optimal degradation rates for replacement of load bearing tissues, silk fibroin has been increasingly investigated as a potential ligament or tendon graft in recent decades [18, 27-31]. A number of researchers have been working on silk based ACL scaffold. Horan and Altman et al. did a study on the architectures of silk matrix and determined the cabled structure would be optimal for ligament reconstruction [32]. A series of additional studies have been performed on hierarchical organization of silk matrix, and a 6-cord wire-rope silk fiber matrix is suggested for ligament regeneration [5, 23, 33]. Many in vitro studies have been performed on silk based scaffold for ligament tissue engineering, to evaluate the effect of surface treatment, biological factors, or cell types on the biological and mechanical behavior for tendon or ligament scaffold [12, 14-16, 21, 34-36]. There are also quite a few studies that have tested the silk based ligament scaffold in the animals. Rabbits, goats and pigs are frequently used animal models for evaluation of in vivo response of silk based ligament scaffolds [14, 17, 37, 38]. For a silk based ACL scaffold named SeriACL a human clinical trial has been conducted in Europe to assess the safety and efficiency for a completely ruptured ACL reconstruction [39]. Thus many promising developments have been achieved for silk based ligament scaffold in previous studies, bringing silk based tissue engineered ACL much closer to widespread clinical application [40, 41].

However, most of the previous studies on ACL scaffolds have only focused on the scaffold itself, largely ignoring the critical connection site of the ACL scaffold to the bone tunnel, which is very important for successful ACL repair. Since it is similar to hamstring autograft reconstruction, the scaffold to bone integration is almost always poor. Bone tunnel expansion can occur, predisposing the scaffold to pullout. To avoid bone tunnel expansion and achieve effective attachment of ACL scaffold into the bone tunnel, sufficient surface contact between scaffold and bone, and suitable biomechanical stimulation are essential for scaffold to bone attachment. Although some fixation methods, such as interference screws, can be adopted to anchor the ACL scaffold into bone tunnel, these methods impose a decidedly non-physiological barrier to healing.

Many approaches have been tried by biomaterial engineers and orthopaedic surgeons to achieve a better biological attachment. The major concern is to provide appropriate cellular cues that result in an effective healing response between e.g. tendon and bone. Due to good properties regarding osteoconductivity and bioresorption, bone cements, such as brushite calcium phosphate cement (CPC) and injectable tricalcium phosphate (TCP), can augment the peri-tendon bone volume and promote bone ingrowth into the healing interface and significantly enhance the tendon-bone integration after tendon or ligament reconstruction [42, 43]. Cell based therapies have also been employed. Since a sufficient amount of stem cells is probably necessary for optimal tissue regeneration, mesenchymal stem cells (MSCs) have been applied as potential agents to enhance tendon healing into the bone tunnel. MSCs coated scaffolds have been reported to develop an interpositional zone of fibrocartilage between tendon and bone during tendon reconstruction, had high quality of osteointegration and perform significantly well on biomechanical testing [44, 45]. Bioactive factors represent another potentially powerful means of promoting tendon to bone healing. The highly osteoinductive properties of bone morphogenetic proteins (BMPs) are now widely recognized, and implemented within daily clinical practice. Endogenous BMP-2 and BMP-7 participate in tendon to bone healing and their functions involve downstream signal transduction mediators. The BMP-2 can enhance bone ingrowth and accelerate the healing process when a tendon scaffold is transplanted into a bone tunnel [46, 47].

But nearly all pre-clinical studies listed above have focused primarily on cell biology aspects (cell sources or osteoinductive/conductive agents) that can applied at the tendon/scaffold to bone interface, and neglect implications of primary mechanical stability. They hope that cells in the bone tunnel might recognize the tendon/scaffold surface as a potentially osteoconductive matrix, promoting rapid bone ingrowth that quickly provides secondary mechanical stability through an improved attachment of tendon to bone.

Few researchers have focused on how an osteoconductive/inductive construct might be used to achieve superior biological healing and secondary stability while also providing adequate primary mechanical stability.

Therefore, the problem motivating the present invention is to provide for a device for fixing a flexible element such as a synthetic or natural ligament or tendon to a bone that is improved concerning mechanical stability and particularly allows for an efficient biological healing at the same time.

This problem is solved by a device having the features of claim 1.

According thereto, the device for fixing a flexible element, particularly in the form of an artificial or natural ligament or a tendon, to a bone, comprises: an insert being designed to hold said flexible element, wherein particularly the flexible element contacts the insert, and an anchor, wherein the insert is designed to be inserted into said anchor, and wherein the anchor is designed to be inserted into a bore hole of said bone together with said insert inserted into the anchor, in order to fix the flexible element to the bone.

Preferably, the insert is formed out of an osteoconductive and/or osteoinductive material or comprises an osteoconductive and/or osteoinductive material.

In this regard, an osteoconductive material is material that is designed to serve as a scaffold or guide for the reparative growth of bone tissue. Osteoblasts from the margin of the bone bore hole utilize such a material as a framework upon which to appropriately spread, migrate, proliferate, and ultimately generate new bone. In this sense an osteoconductive material may be regarded as a “bone compatible” material.

Further, an osteoinductive material is a material that is designed to stimulate osteoprogenitor cells to preferentially differentiate into osteoblasts that then begin new bone formation. An example for such osteoinductive cell mediators are bone morphogenetic proteins (BMPs), and tri-calcium phosphate bearing biomaterials. Thus, an insert that is osteoconductive and osteoinductive will not only serve as a scaffold for currently existing osteoblasts but will also trigger the formation of new osteoblasts, and thus allows for faster integration of the insert into the bone.

The described invention allows one to adequately provide for robust initial mechanical stability due to the anchor, while at the same time promoting contact between the insert/flexible element and the walls of the bore hole or bone tunnel can be established that promotes the afore-mentioned biological healing, e.g. ingrowth of the bone into the insert.

According to an embodiment of the invention, the anchor is designed to be inserted into said bore hole (also denoted as bone tunnel) of the bone along an insertion direction together with said insert inserted into the anchor, wherein the insert is preferably designed to be inserted into the anchor counter to said insertion direction.

According to an embodiment of the invention, the anchor comprises a head part and a first and a second leg facing each other, wherein said legs preferably protrude from said head part along the insertion direction. Particularly, the legs are integrally formed with the head part. Further, the anchor is designed to be inserted into the bore hole of the bone with the legs ahead so that the head part is particularly flush with the surface region of the bone around the bore hole.

In an embodiment of the invention, particularly for the use with synthetical flexible elements (e.g. ligaments or tendons, particularly ACL scaffolds), the head part comprises an annular shape, wherein particularly the head part comprises a central opening designed for passing through said flexible element.

In an alternative embodiment, particularly for the use with natural flexible elements (e.g. ligaments or tendons, particularly autografts), the head part comprises two opposing cut-outs designed for receiving/bypassing the flexible element, wherein each cut-out is formed in a boundary region of the head part extending from one leg to the other.

According to a further aspect of the invention, the insert is preferably arranged between the legs of the anchor when the insert is inserted into the anchor as intended.

For proper insertion of the insert into the anchor, the insert preferably comprises a first and a second guiding recess according to a further embodiment of the invention, wherein these recesses are preferably designed to receive the legs of the anchor in a form fitting manner when the insert is inserted into the anchor.

Preferably, each guiding recess is delimited by a surface of the insert forming the bottom of the respective guiding recess, wherein the two surfaces face away from each other, and two opposing boundary regions protruding from the respective surface and extending along the insertion direction forming the side walls of the respective guiding recess. In a variant of the invention, the two surfaces are convex, i.e. bulged towards the respective leg that slides along the surface of the associated guiding recess upon insertion of the insert into the anchor.

Further, each of said boundary regions preferably comprises a contact surface being designed to contact the bone when the anchor is inserted into the bore hole of the bone together with the insert as intended, which contact surface extends along the respective guiding recess. In this way, bone cell ingrowth into the insert around which the flexible element is laid is achieved that finally results in a bone firmly holding the flexible element. Further, also the anchor comprises an outside for contacting the bone, wherein preferably said outside comprises a toothed surface in order to increase friction between the outside of the anchor and the walls of the bore hole. Particularly, the contact surfaces of the boundary regions of the insert are essentially flush with said outside of the anchor when the insert is inserted into the anchor as intended. Hence, while the outside of the anchor serves for mechanical stability right from the start, the contact surfaces of the insert are designed to promote biological healing and thus provide additional stability in the long term.

To further increase mechanical stability, the insert is at least in sections tapered in a variant of the invention, so that upon inserting the insert into the anchor, said surfaces of the insert press the legs away from each other, wherein particularly the anchor is designed to be inserted into the bore hole in the insertion direction with the insert being inserted into the anchor in a first position, in which the insert is not fully inserted into the anchor, wherein the insert is designed to be pulled into a second position counter to the insertion direction when the anchor is inserted into the bore hole of the bone as intended, in which second position the insert is fully inserted into the anchor and thus presses the legs against the wall of the bore hole.

According to a further aspect of the invention, the legs preferably comprise an inner surface, wherein the two inner surfaces face each other, and wherein particularly said inner surfaces are concave so as to match with the surface of the respective guiding recess, i.e., each inner surface is preferably designed to slide along the surface of the respective guiding recess when inserting the insert into the anchor, and to rest on the associated surface of the insert thereafter. Further, each leg preferably comprises two lateral surfaces coming off the respective inner surface, wherein particularly the lateral surfaces of a leg face away from each other, and wherein particularly each lateral surface rests on an associated boundary region, when the insert is inserted into the anchor as intended. Further, each lateral surface preferably encloses an angle of particularly 45° with an extension plane along which the respective leg extends.

Particularly, according to a further aspect of the invention, the insert comprises a first wall region and a second wall region, wherein particularly the first guiding recess is formed in the first wall region, and wherein particularly the second guiding recess is formed in the second wall region. Preferably, the two wall regions are integrally connected by a connecting region of the insert, which connecting region preferably comprises a concave surface.

Further, for receiving the flexible element, the insert preferably comprises a groove or an open channel, wherein particularly said groove is formed by the two wall regions and the connecting region. The groove is preferably formed such that the flexible element can be laid around the connecting region and is then arranged at least in sections in said groove tightly contacting the insert.

In case the head part of the anchor comprises an annular shape with a central opening, the flexible element passes through the opening of the head part when the insert is inserted into the anchor as intended and when the flexible element is arranged with respect to anchor and insert as intended.

Alternatively, in case the head part comprises said two opposing cut-outs, the flexible element preferably extends through the cut-outs of the head when the insert is inserted into the anchor as intended and when the flexible element is arranged with respect to anchor and insert as intended.

As mentioned before, the flexible element may be a natural ligament or a natural tendon.

Particularly, the flexible element is a synthetic ligament or tendon, particularly an anterior cruciate ligament (ACL) scaffold.

According to a further embodiment of the present invention, such a flexible element comprises two twisted cords, wherein particularly the cords have a turn every 12 mm. Further each cord comprises 144 twisted yarns, wherein particularly the yarns have a turn every 10 mm. Each yarn comprises two twisted bundles, wherein particularly each bundle has a turn every 2 mm. Finally, each bundle comprises 6 fibres, which fibres preferably comprise fibroin, e.g. silk.

In this regard, fibroin in the sense of the invention refers in particular to a polypeptide, which consists of layers of antiparallel beta-sheets and is particularly characterized by a recurrent amino acid sequence, wherein the recurrent amino acid sequence is Gly-Ser-Gly-Ala-Gly-Ala. Non-limiting examples for fibroin include Bombyx mori fibroin with a light chain (UniProt. P21828) and a heavy chain (UniProt. P05790), and Bombyx mandarina fibroin comprising a heavy chain (Q99050). UniProt. numbers refer to entries in the Universal Protein Knowledgebase (http://www.uniprot.org/).

According to a further alternative embodiment of the present invention, the flexible element comprises three braided cords, wherein particularly the cords have a turn every 12 mm. Further, each cord comprises 96 twisted yarns, wherein particularly the yarns have a turn every 10 mm. Each yarn comprises two twisted bundles, wherein particularly each bundle has a turn every 2 mm. Finally, each bundle comprises again 6 fibres, which fibres preferably comprise fibroin, e.g. silk (see also above).

According to a further embodiment of the present invention, the insert comprises one of the following substances: tricalcium phosphate (Ca₃(PO₄)₂), hydroxylapatite (Ca₁₀(PO₄)₆(OH)₂), calcium phosphate, particularly as a component of a of bone cement, calcium silicate (Ca₂SO₄), particularly as a component of a bone cement, or silicate-substituted calcium phosphate or other osteoinductive/osteoconductive bioceramics/bioglasses.

Further, according to yet another embodiment of the present invention, the anchor comprises one of the following substances: polyether ether ketone (PEEK), poly lactic acid, poly(lactic-co-glycolic acid) (PLGA), poly-ε-caprolactone (PCL), titanium-based alloy, or magnesium-based alloy. The anchor may also comprise or may be formed out of another biopolymer or implantable metal.

According to another aspect of the present invention, a tool set is provided for inserting a device according to the invention into a bore hole or bone tunnel.

According to claim 28 such a tool set comprises at least a first tool for pressing the device into said bore hole, wherein said first tool comprises an elongated shaft having a free end that is designed to engage with the anchor, particularly with the head part of the anchor, for pressing the device into said bore hole or bone tunnel, wherein said elongated shaft further comprises a groove for receiving the flexible element extending from the anchor/insert upon insertion of the device into the bore hole of the bone.

In a variant of the invention, the first tool comprises at its free end a plurality of protrusions (particularly three protrusions) that are designed to engage with corresponding recesses formed in the head part of the anchor, particularly in a periphery of the opening of the annular head part.

In a further variant of the first tool the free end is shaped hollow cylindrical and comprises a discontinuation extending along the longitudinal axis of the shaft corresponding to said groove of the shaft. Wherein the shaft preferably comprises a step at the free end such that the free end has a reduced outer diameter compared to the remaining shaft, wherein the cylindrical free end is designed to engage in a form fitting manner with said opening of the annular head part of the anchor for pressing the anchor into the bore hole of the bone.

Further, the tool set may comprise a second tool comprising a handle and a drill sleeve protruding from a free end of the handle for guiding a drill for drilling said bore hole into the bone, wherein a free end of the drill sleeve may be tapered or sharpened for assuring a good grip on the bone while pressing the free end of the drill sleeve against the bone.

Further, the tool set may comprise a third tool for positioning the second tool, wherein the third tool comprises a first leg extending along an extension direction, as well as a second and a third leg extending from opposite ends of the first leg so that particularly a u-shaped or arc-shaped body of the third tool is formed, wherein a plug protrudes from a free end of the third leg along the extension direction for insertion into the bore hole of the bone (e.g. into the distal femur in case the flexible element replaces the anterior cruciate ligament, for instance). Further, the second leg opposing the third leg preferably comprises a through-opening aligned with said plug, so that when the plug is inserted into the bore hole of the bone (e.g. distal femur), the second tool can be inserted with its drill sleeve into the through-opening of the second leg, so that a bore hole (e.g. tunnel) can be drilled into another bone (e.g. the tibia in case the flexible element replaces the anterior cruciate ligament for instance) in axial alignment with the bore hole of said bone (e.g. distal femur). Then, the free end of the flexible element distal to the anchor/insert can be passed through said bore hole or tunnel of the further bone (e.g. tibia) and fixed to said further bone, for instance by means of an interference screw.

Finally, another aspect of the present invention is to provide for a method for inserting a device according to the invention into a bore hole of a bone, particularly using said tool set, wherein the method comprises the steps of drilling a bore hole into a bone, particularly into the distal femur, and pressing the anchor with inserted insert with the legs of the anchor ahead into said bore hole in an insertion direction, wherein particularly the insert is fully inserted into the anchor upon inserting the anchor into the bore hole or wherein particularly the insert is inserted into the anchor in a first position, in which the insert is not fully inserted into the anchor, wherein, when the anchor is inserted into the bore hole of the bone as intended, the insert is pulled into a second position counter to the insertion direction by means of the flexible element, in which second position the insert is more or fully inserted into the anchor and the legs of the anchor are pressed against a wall of the bore hole of the bone by means of the insert.

According to a further aspect of this method, before drilling said bore hole, a small lateral incision is made in the knee to put an endoscope into the knee joint.

According to a further aspect of the method, a trans-tibial bone tunnel is then drilled, as well as said bore hole in the distal femur, wherein particularly said bone tunnel and said bore hole preferably have a diameter in the range from 4 mm to 8 mm, particularly 7 mm, and wherein particularly said bore hole has a depth of 15 to 30 mm, particularly 20 mm, wherein said bone tunnel and said bore hole are particularly drilled such that said bone tunnel is aligned with said bore hole.

According to a further aspect of the method, the knee is then bent, and a medial incision is made.

According to a further aspect of the method, said bore hole is then preferably enlarged to a diameter in the range from 7 mm to 12 mm, particularly 9 mm, particularly through said medial incision.

According to a further aspect of the method, the insert is then inserted (e.g. as described above) into said bore hole, particularly by means of the first tool, through the medial incision.

According to a further aspect of the method, a free end of the flexible element is then pulled through the trans-tibial bone tunnel.

According to a yet further aspect of the method, the flexible element is then pulled tight, wherein the tension is particularly adjusted by the surgeon, and fixed with a fixing element, particularly with an interference screw (φ6×19 mm), to the tibia, wherein said fixing element is particularly screwed into the trans-tibial bone tunnel.

In the following, an alternative variant of the method is described.

According to an aspect of this alternative method, a longitudinal medial skin incision is made, particularly approximately 5 cm proximal to the superior margin of the patella to the tibial tubercle.

According to a further aspect of the alternative method, the knee joint is then accessed with a medial parapatellar capsular approach.

According to a further aspect of the alternative method, the native ACL is cut and removed.

According to a further aspect of the alternative method, said bore hole, particularly of diameter 9 mm, is then drilled over the footprint of ACL in the femur, particularly 20 mm in depth.

According to a further aspect of the alternative method, particularly to avoid damage to the articular cartilage on the medial condyle, the drilling direction is adjusted to 11 o'clock on the transversal plane, and 45° anterior deviation on the sagittal plane using the femoral axes as frame of reference.

According to a further aspect of the alternative method, the second tool is used to guide the drill used for drilling said bore hole, particularly so as prevent slipping and/or wobbling of said drill.

According to a further aspect of the alternative method, a trans-tibial bone tunnel, particularly 7.0 mm in diameter, is then drilled along the axis of said bore hole in the distal femur, wherein particularly the third tool is used to guide the drill used for drilling said further tunnel into the tibia.

According to a further aspect of the alternative method, the insert is inserted (e.g. as described above) into said bore hole, particularly by means of the first tool.

According to a further aspect of the alternative method, a free end of the flexible element is then pulled through the trans-tibial bone tunnel.

According to a further aspect of the alternative method, the knee joint is then flexed to 150°.

According to a yet further aspect of the alternative method, the flexible element is then pulled tight, wherein the tension is particularly adjusted by the surgeon, and fixed with a fixing element, particularly with an interference screw (φ6×19 mm), to the tibia, wherein said fixing element is particularly screwed into the trans-tibial bone tunnel.

Further features and advantages of the invention shall be described by means of detailed descriptions of embodiments with reference to the Figures, wherein

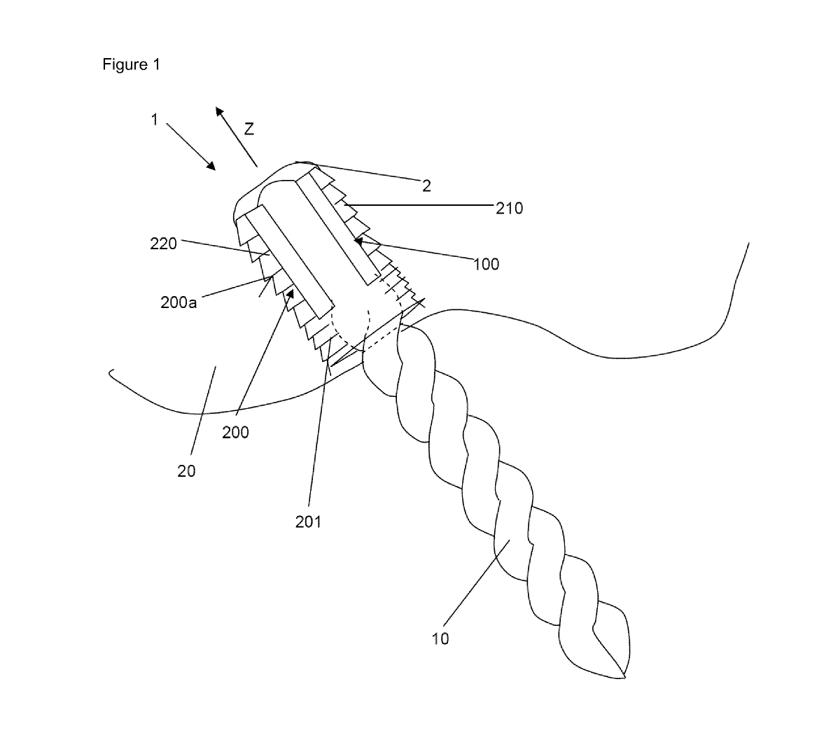

FIG. 1 shows a schematical, partly cross sectional view of a device according to the invention inserted into a bore hole in a bone;

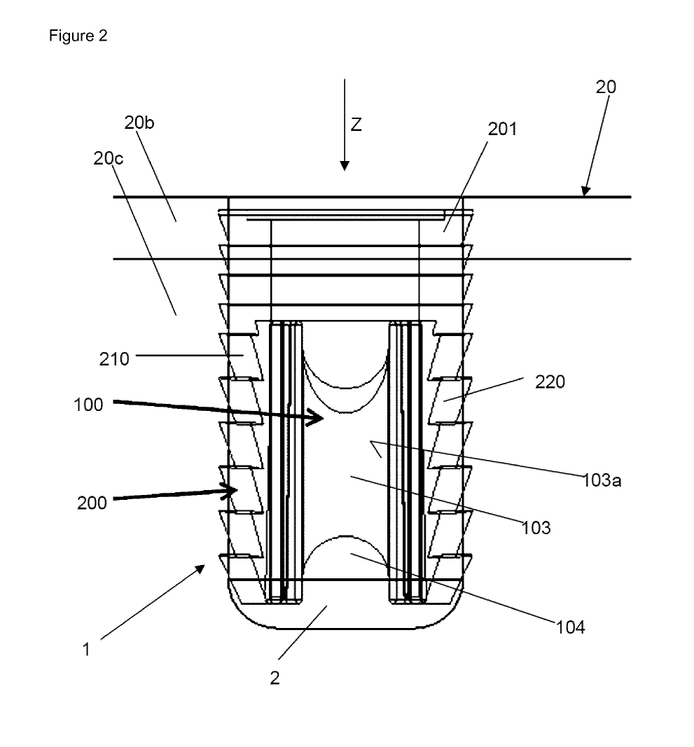

FIG. 2 shows a lateral view of an anchor and an insert of the device according to the invention inserted into a bore hole of a bone for use with a synthetical flexible element (for instance ACL scaffold);

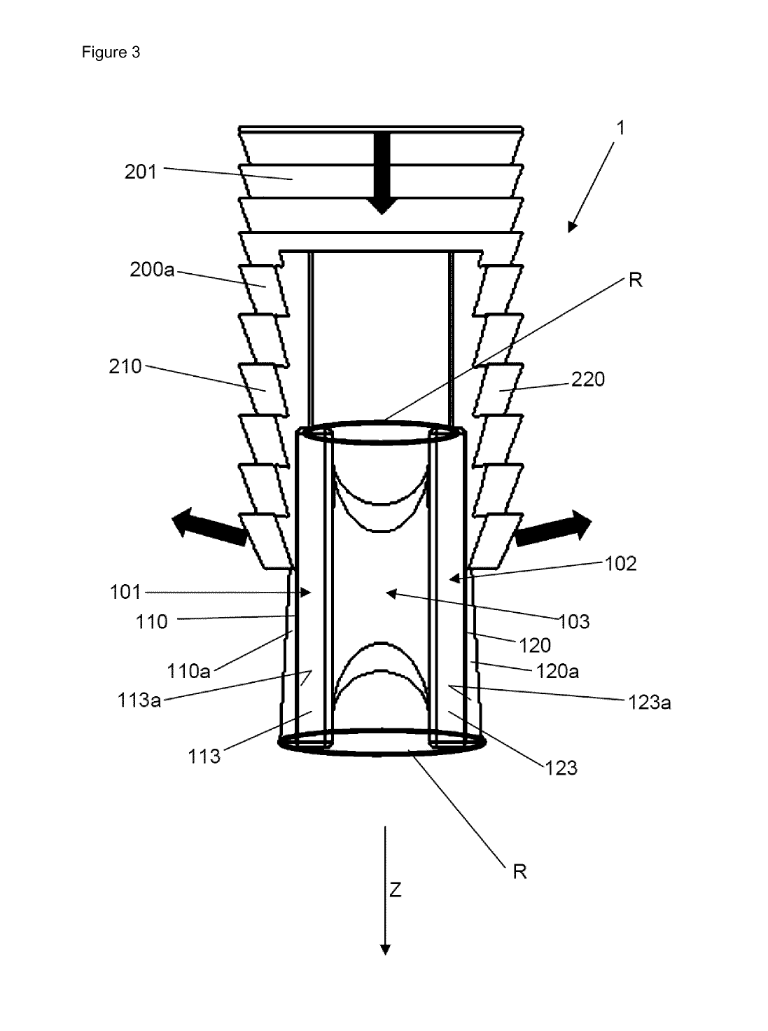

FIG. 3 shows a lateral view of the insert and the anchor of the device according to the invention upon insertion of the insert into the anchor;

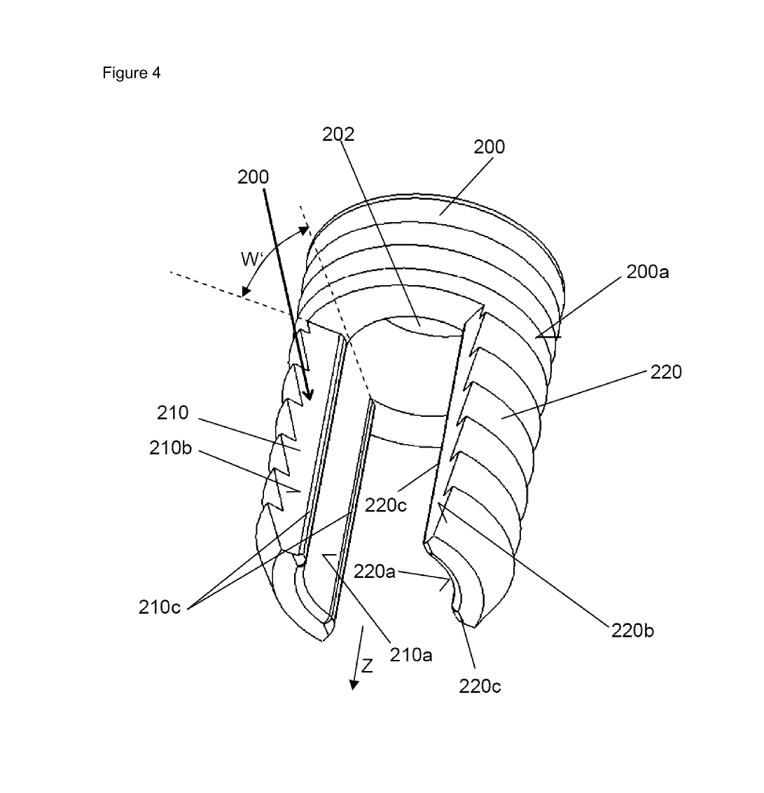

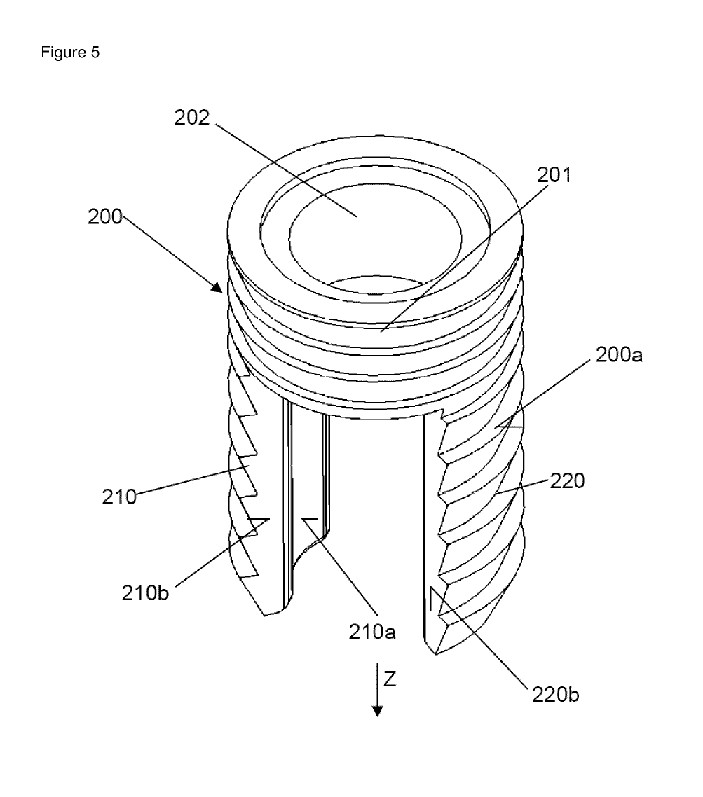

FIGS. 4-5 show perspective views of the anchor shown in FIGS. 1 to 3;

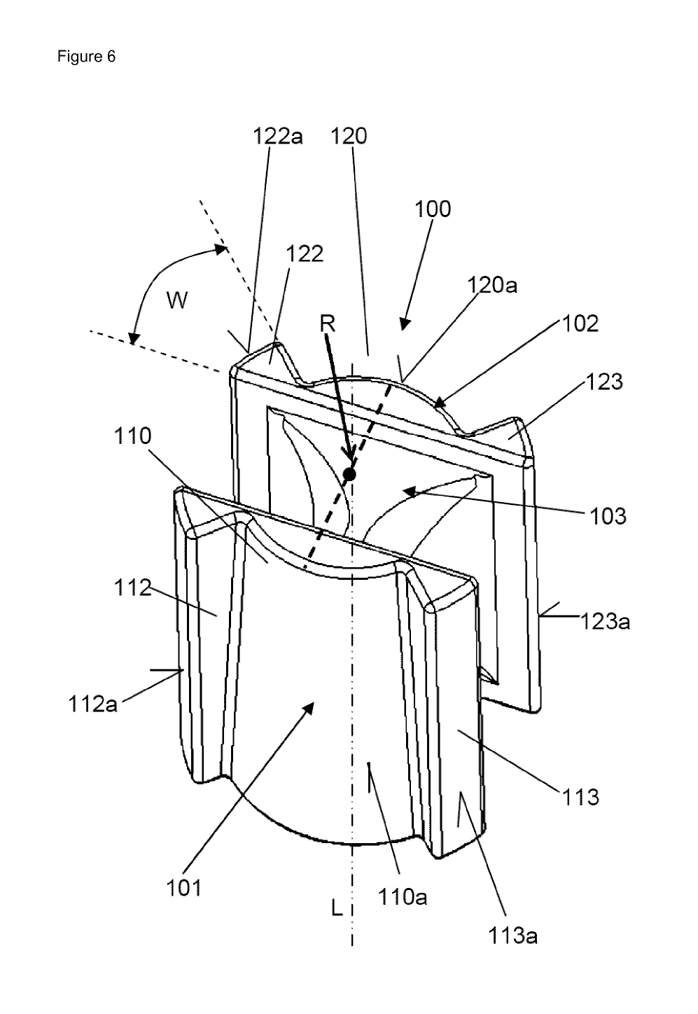

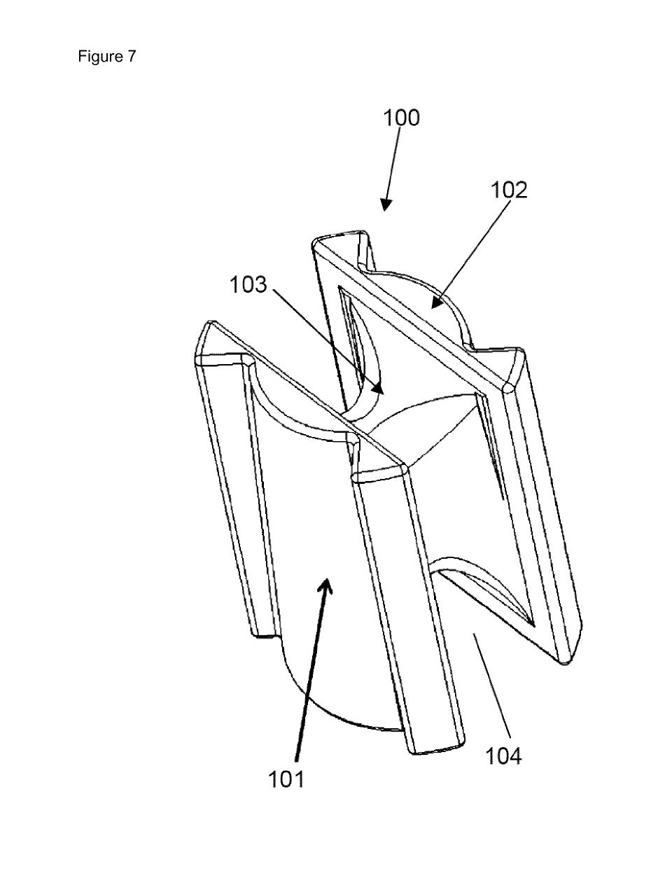

FIGS. 6-7 show perspective views of the insert shown in FIGS. 1 to 3;

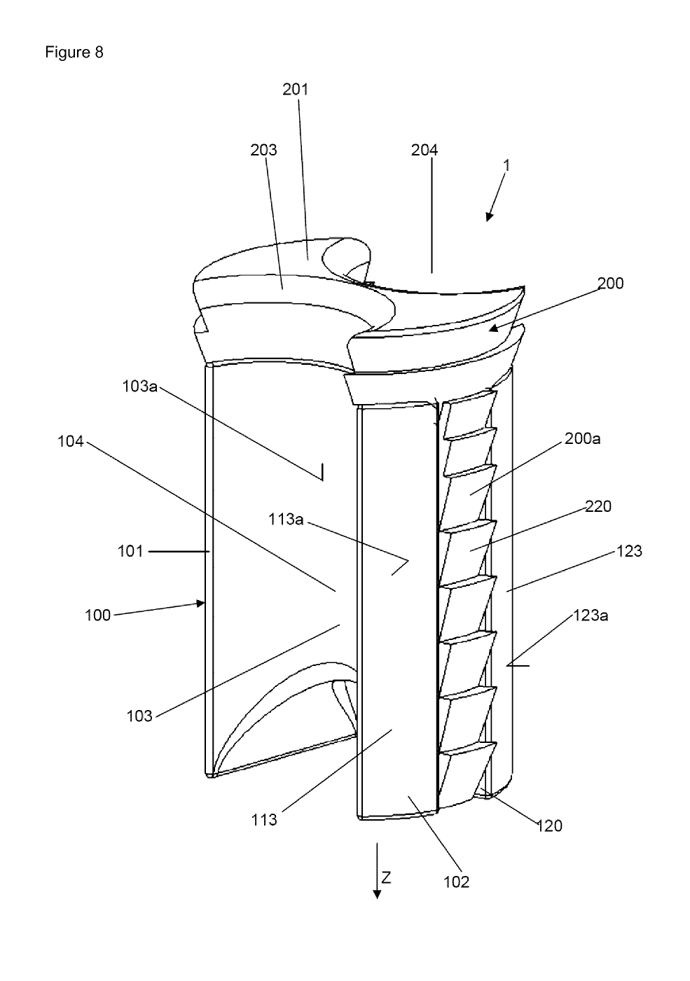

FIG. 8 shows a perspective view of an alternative embodiment of the device according to the invention for fixation of a natural flexible element (e.g. autograft) to a bone;

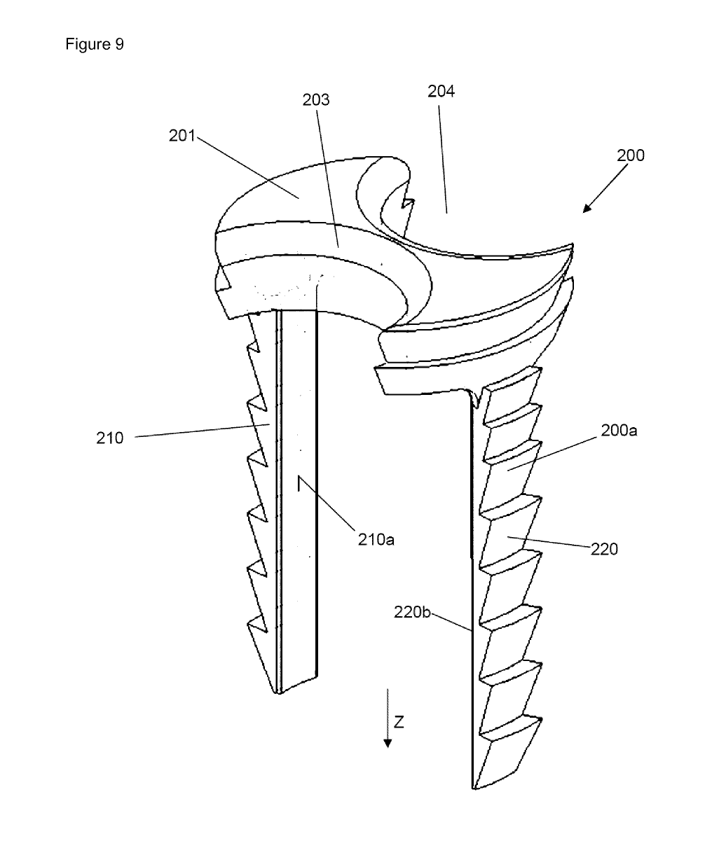

FIG. 9 shows a perspective view of an anchor of the device shown in FIG. 8;

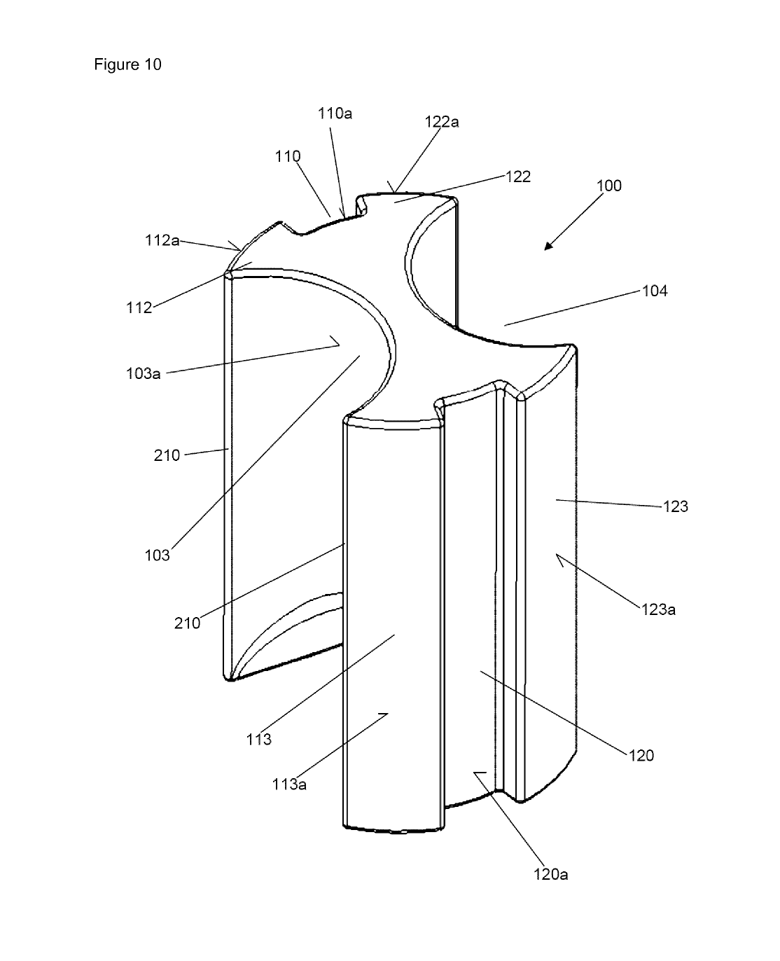

FIG. 10 shows a perspective view of an insert of the device shown in FIG. 8;

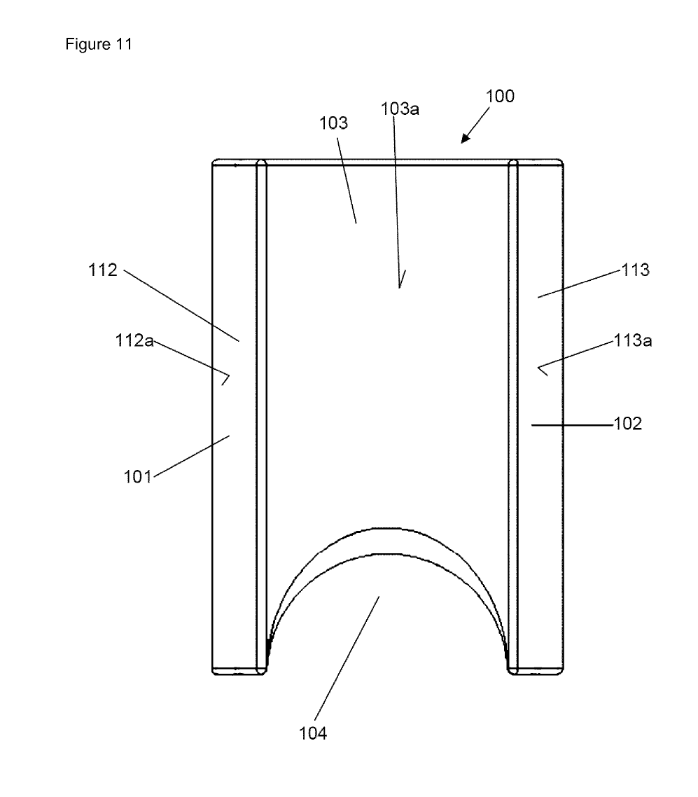

FIG. 11 shows a lateral view of the insert of the device shown in FIG. 8;

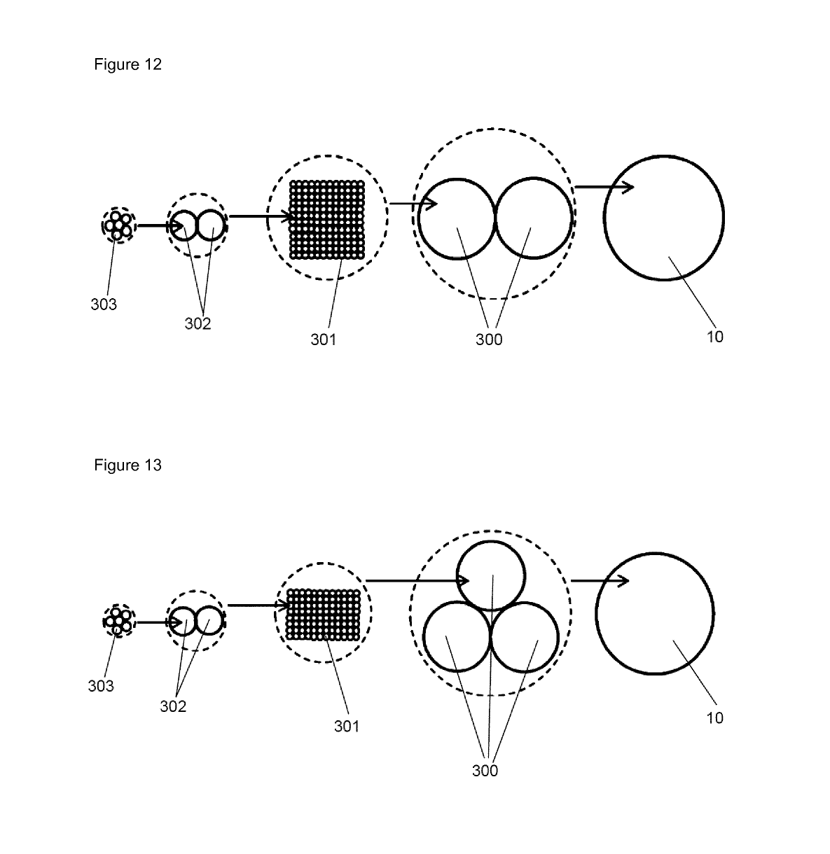

FIG. 12 shows a schematical illustration of the structure of an embodiment of a synthetical flexible element (e.g. ACL scaffold);

FIG. 13 shows a schematical illustration of the structure of an alternative embodiment of a synthetical flexible element (e.g. ACL scaffold);

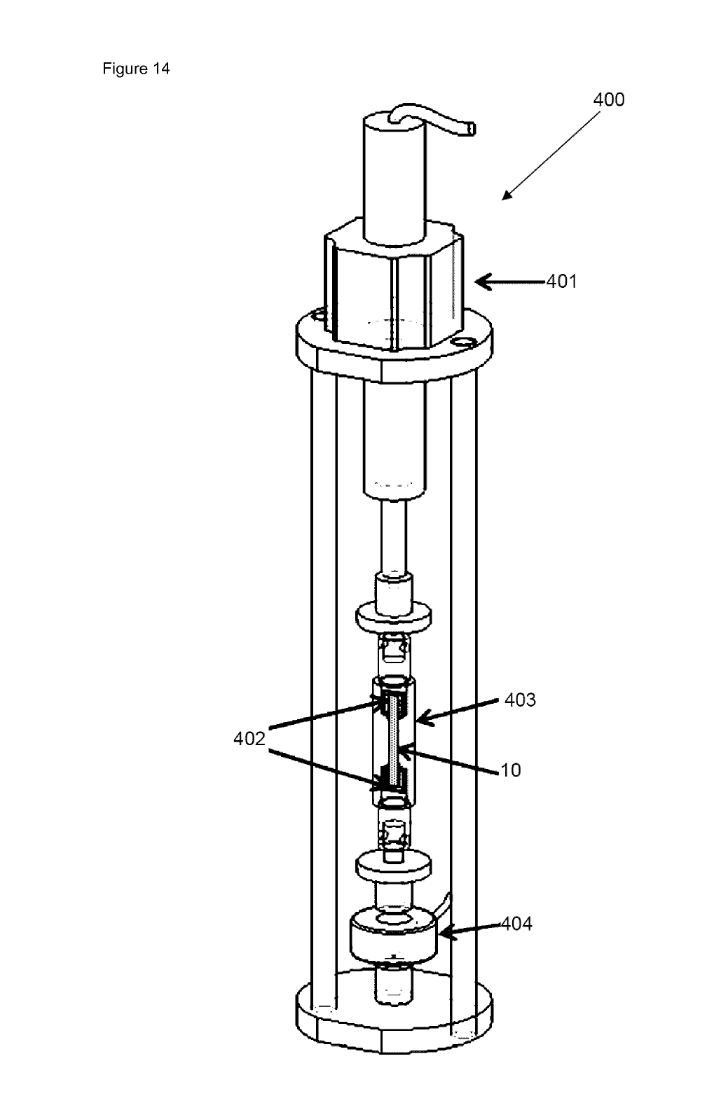

FIG. 14 shows a perspective view of a bioreactor for simulating long term loading of a flexible element (e.g. ligament);

FIG. 15 illustrates a method for inserting a device according to the invention into the femur, particularly for ACL reconstruction;

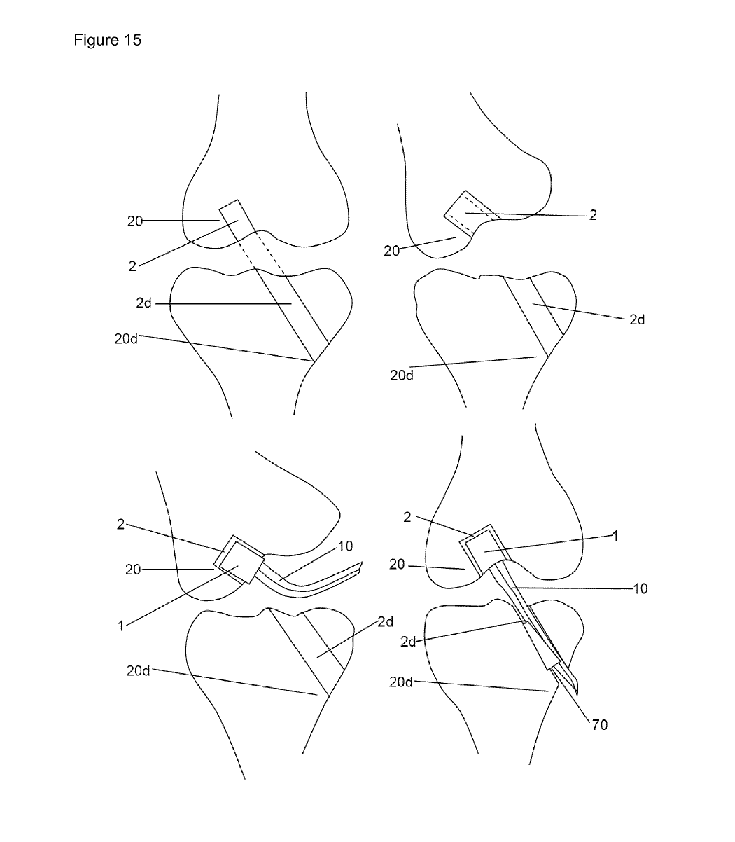

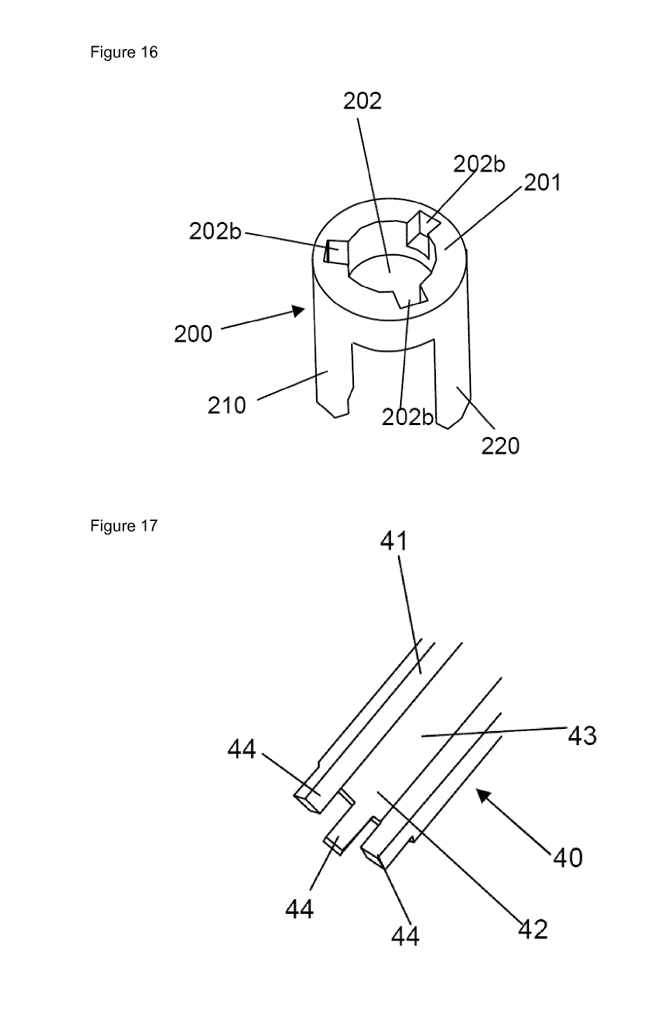

FIG. 16 shows a perspective view of a head part of an anchor of a device according to the invention;

FIG. 17 shows a portion of a first tool for engaging with the head part shown in FIG. 16 for pressing the device according to the invention into a bore hole of a bone;

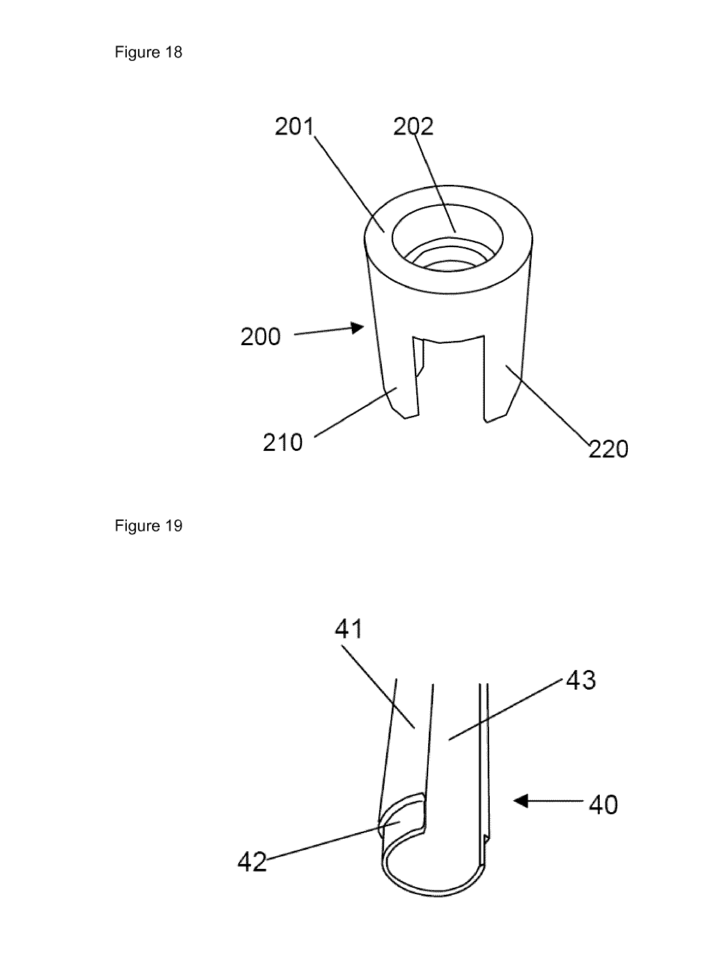

FIG. 18 shows a perspective view of an alternative head part of an anchor of a device according to the invention;

FIG. 19 shows a portion of an alternative first tool for engaging with the head part shown in FIG. 18 for pressing the device according to the invention into a bore hole of a bone;

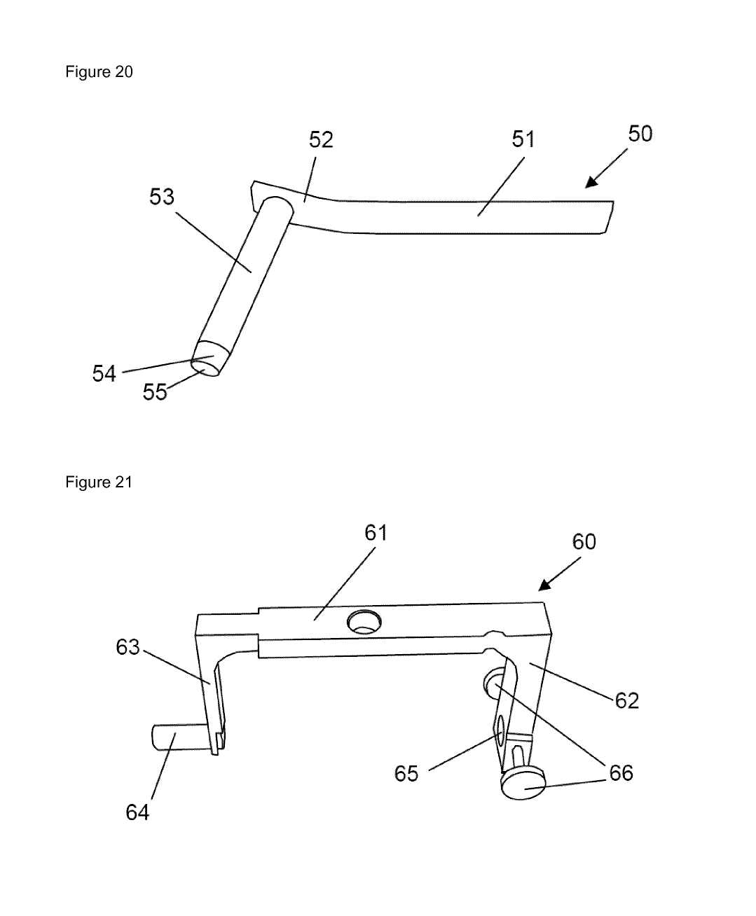

FIG. 20 shows a perspective view of a second tool providing a drill sleeve for guiding a drill being used for drilling a bore hole for insertion of the device according to the invention;

FIG. 21 shows a perspective view of a third tool by means of which the second tool can be positioned in order to drill a further bore hole/tunnel into a further bone so that the further bore hole/tunnel is in axial alignment with the bore hole for the device according to the invention;

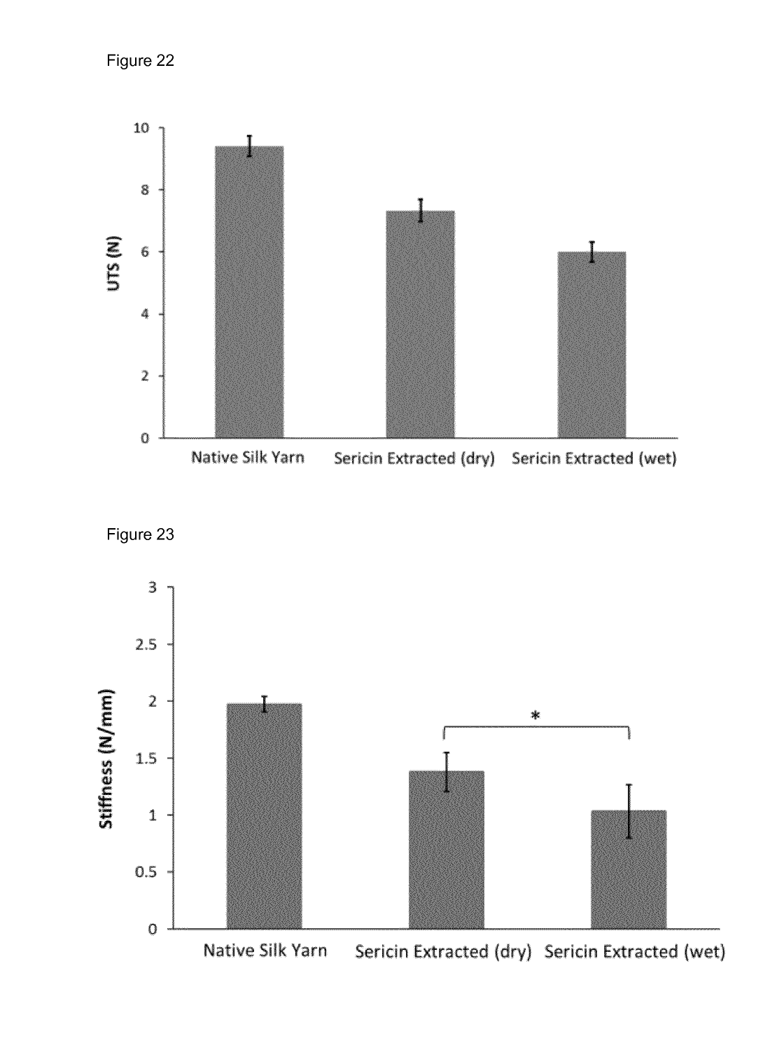

FIG. 22 shows the ultimate tensile strength (UTS) of silk yarn in different conditions;

FIG. 23 shows the stiffness of silk yarn in different conditions;

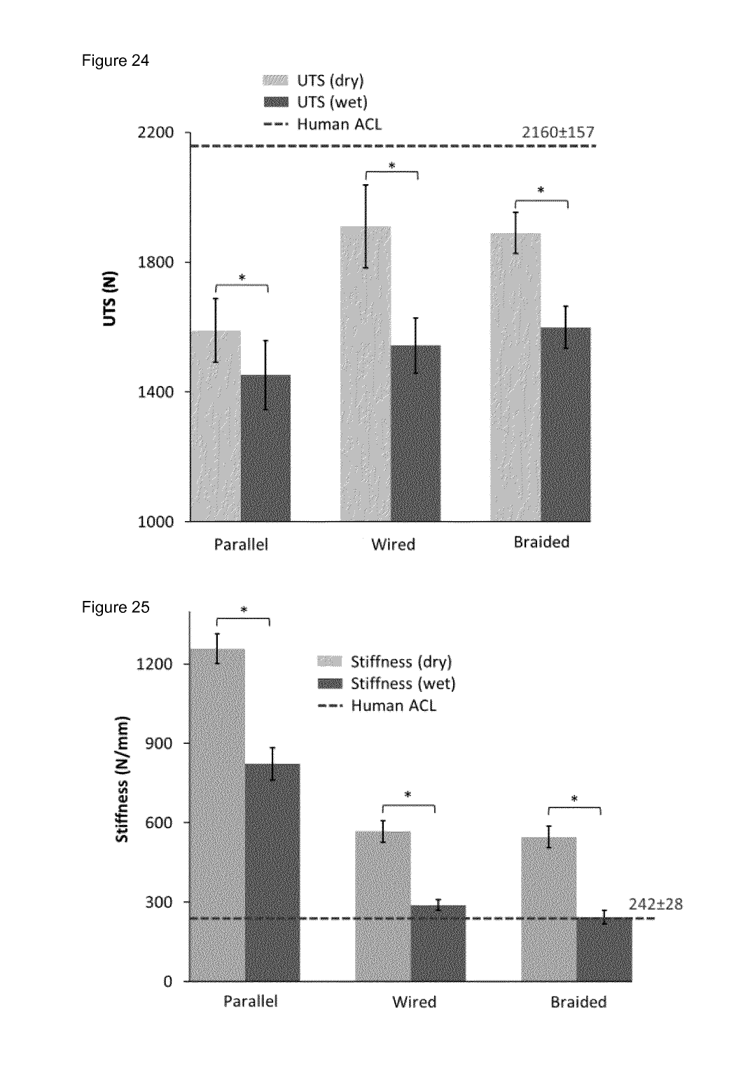

FIG. 24 shows the UTS of flexible elements in the form of silk scaffolds with three architectures (Human ACL value [51]);

FIG. 25 shows the stiffness of flexible elements in the form of silk scaffolds with three architectures (Human ACL value [51]);

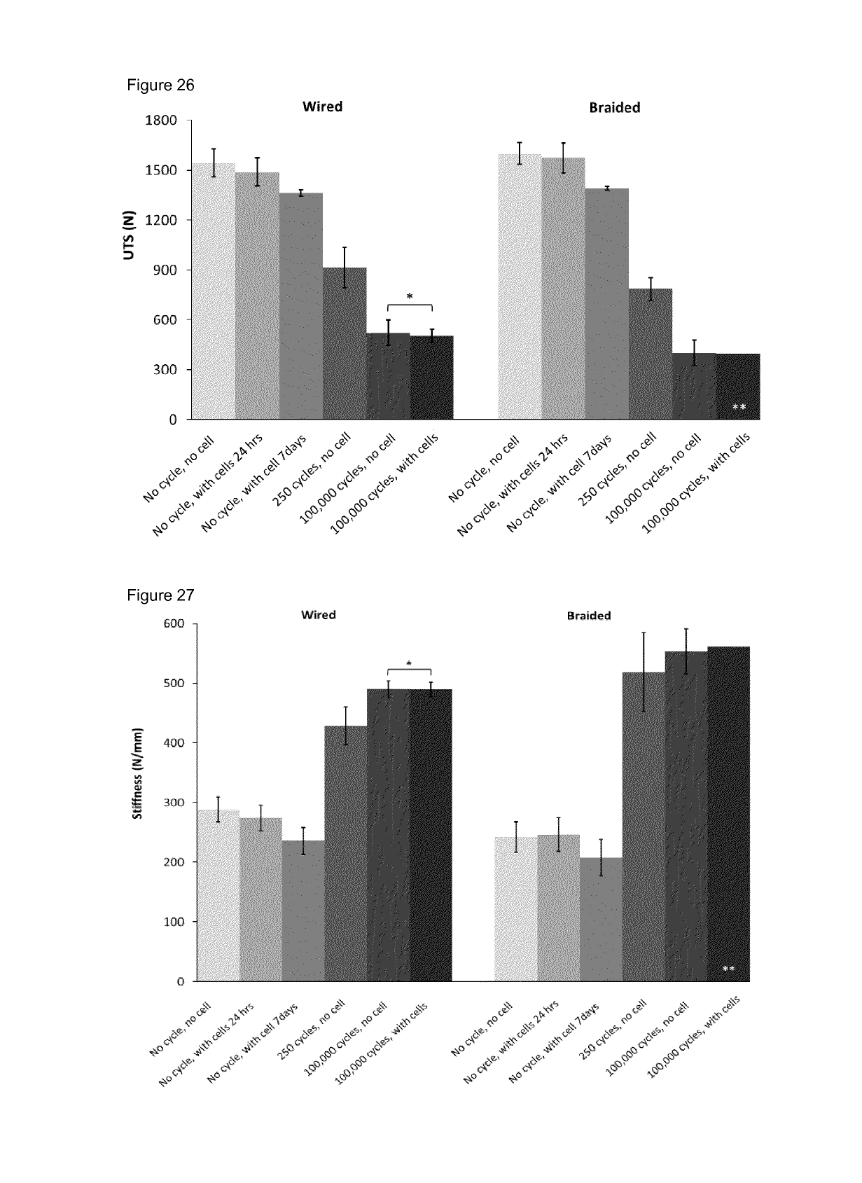

FIG. 26 shows the UTS of flexible elements in the form of wired and braided silk scaffolds under different loading conditions;

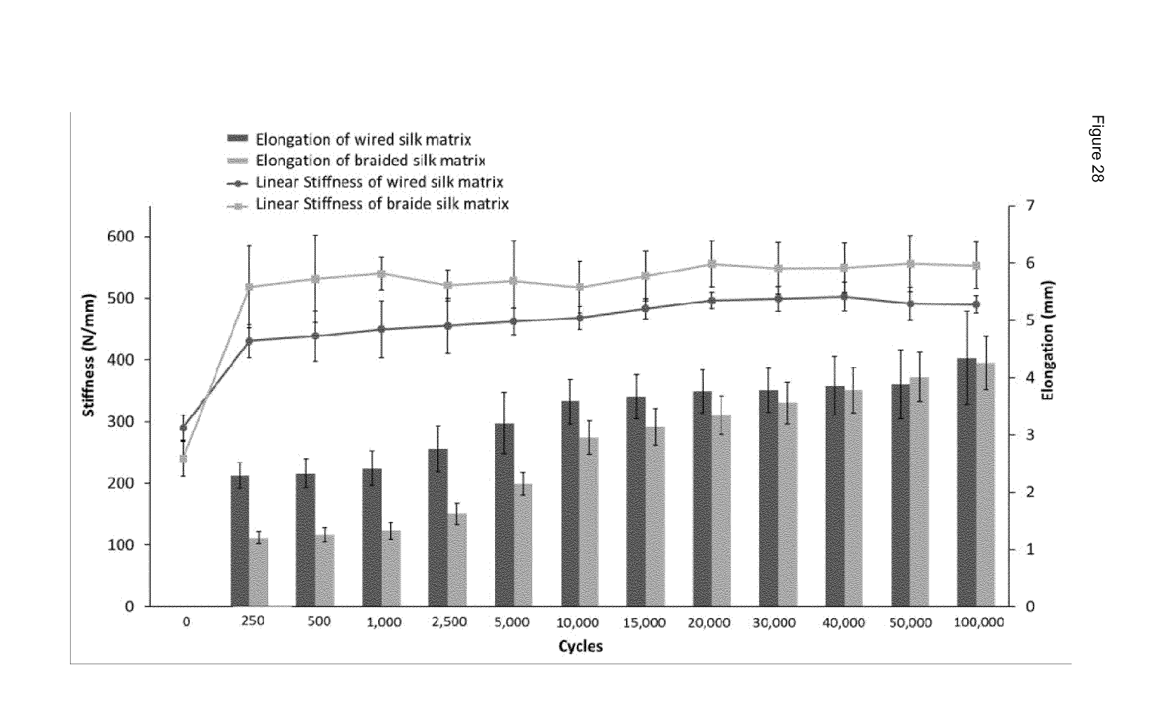

FIG. 27 shows the stiffness of flexible elements in the form of wired and braided silk scaffolds under different loading conditions;

FIG. 28 shows the linear stiffness and elongation of flexible elements in the form of wired and braided silk ACL scaffolds under high cyclic loading;

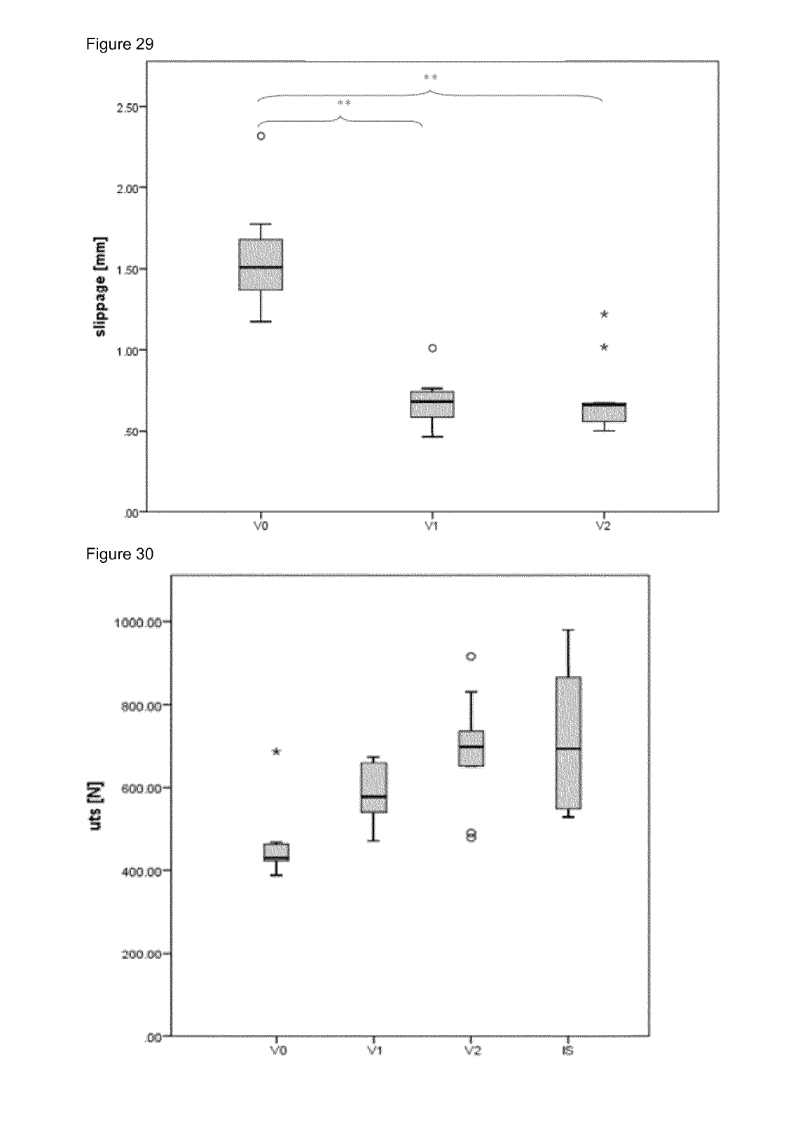

FIG. 29 shows the slippage of the device according to the invention in pig bone for different insert/anchor configurations V0, V1 and V2 of the device according to the invention;

FIG. 30 shows the UTS of the configurations shown in FIG. 29;

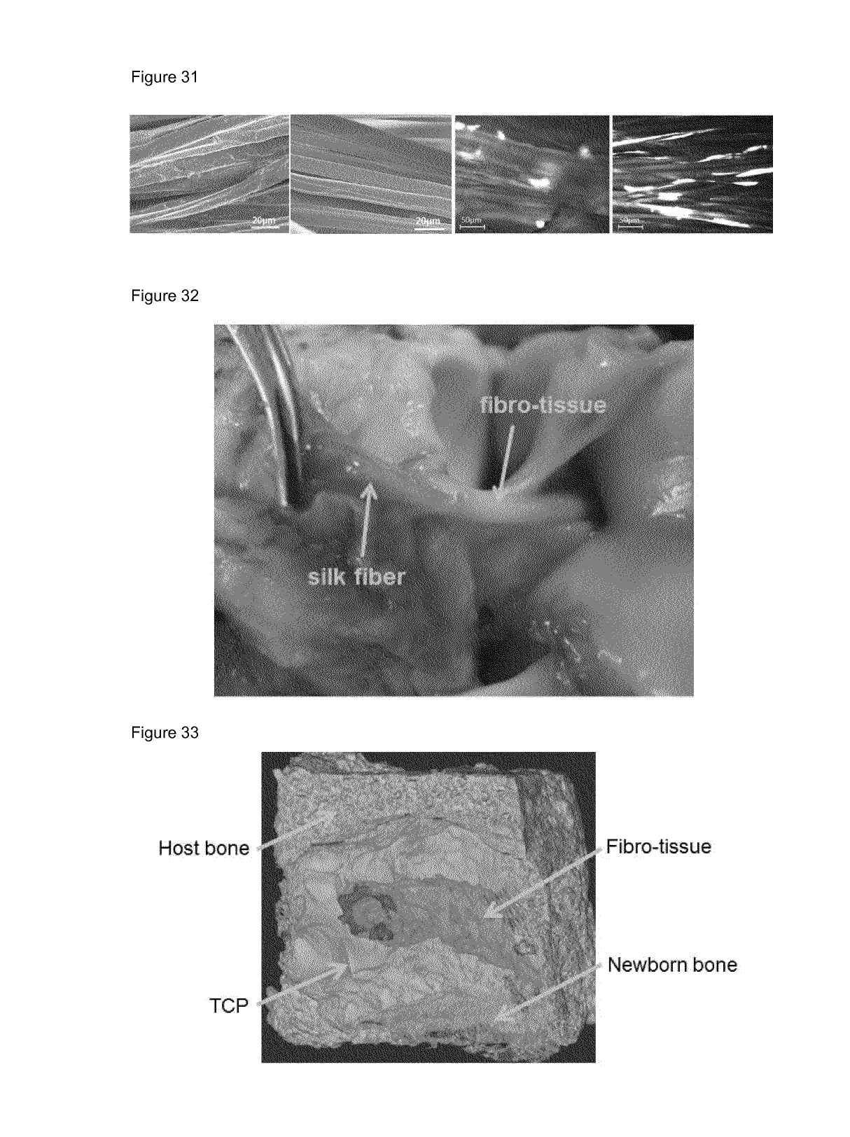

FIG. 31 shows microscope images of silk fibers (from left to right: original raw silk fibers, sericin-extracted silk fibers, fluorescine images at 30 minutes, fluorescine images at 24 hours);

FIG. 32 shows the results of the pilot study (in vivo);

FIG. 33 shows a micro-CT image of regenerated fibro-tissue (pilot study);

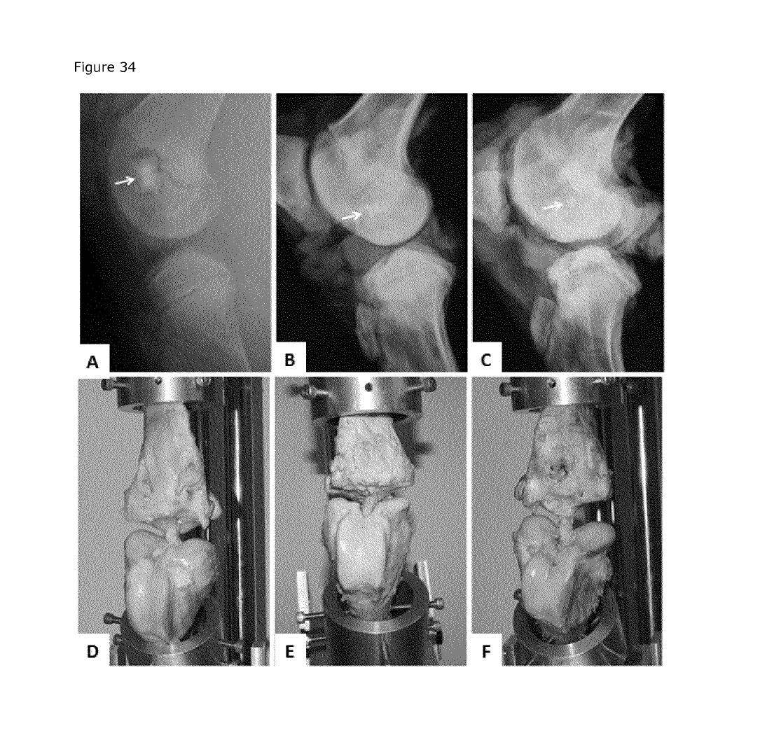

FIG. 34 shows X-ray images of the knee with reconstructed ACL, at different postoperative time points. (A: Day one; B: Three months; C: Six months; D: Native ACL; E: Regenerated ACL at three months; F: Regenerated ACL at six months);

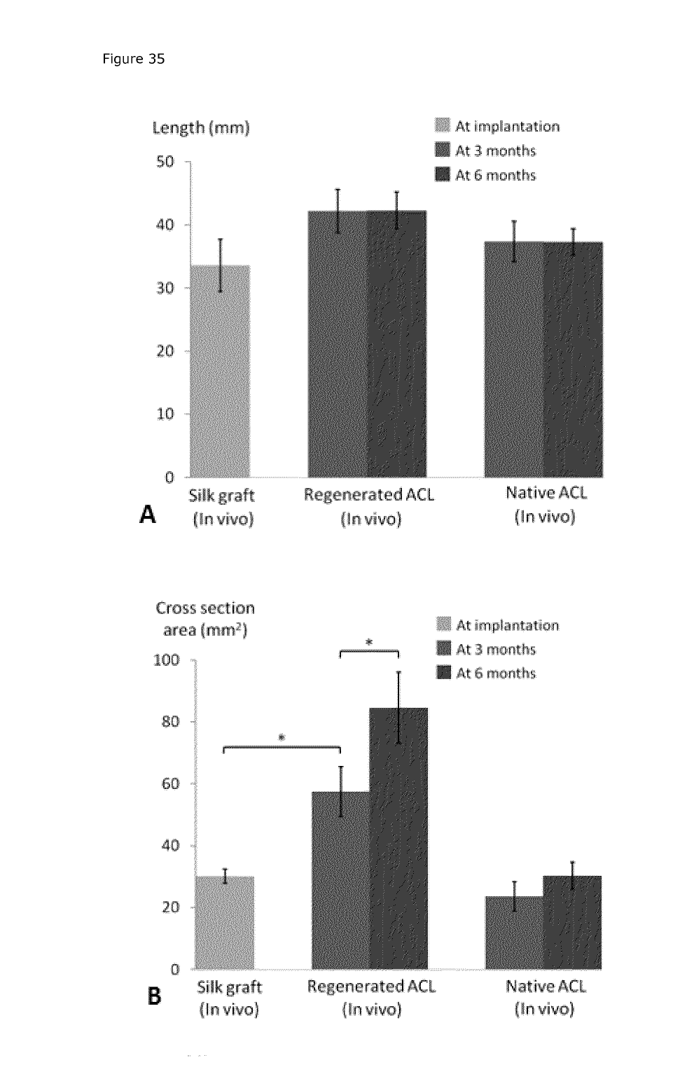

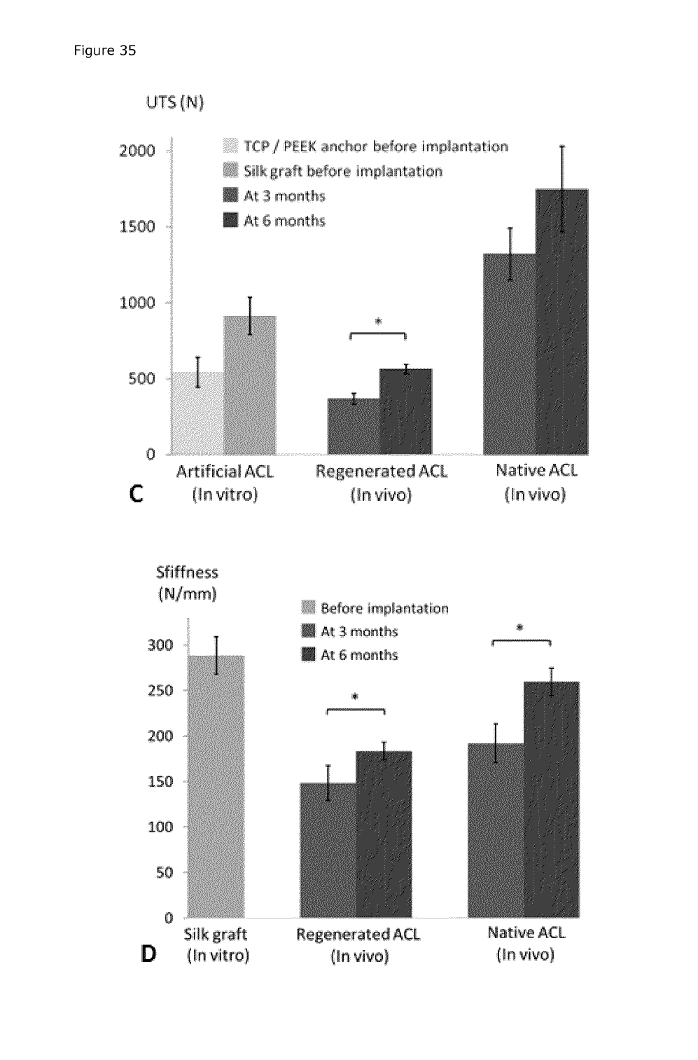

FIG. 35 shows comparison of geometry and mechanical properties of the construct properties at the time of implantation, against the regenerated ACL and native ACL at different time points. (* indicates p<0.05; A: Length; B: Cross section area; C: UTS; D: Stiffness);

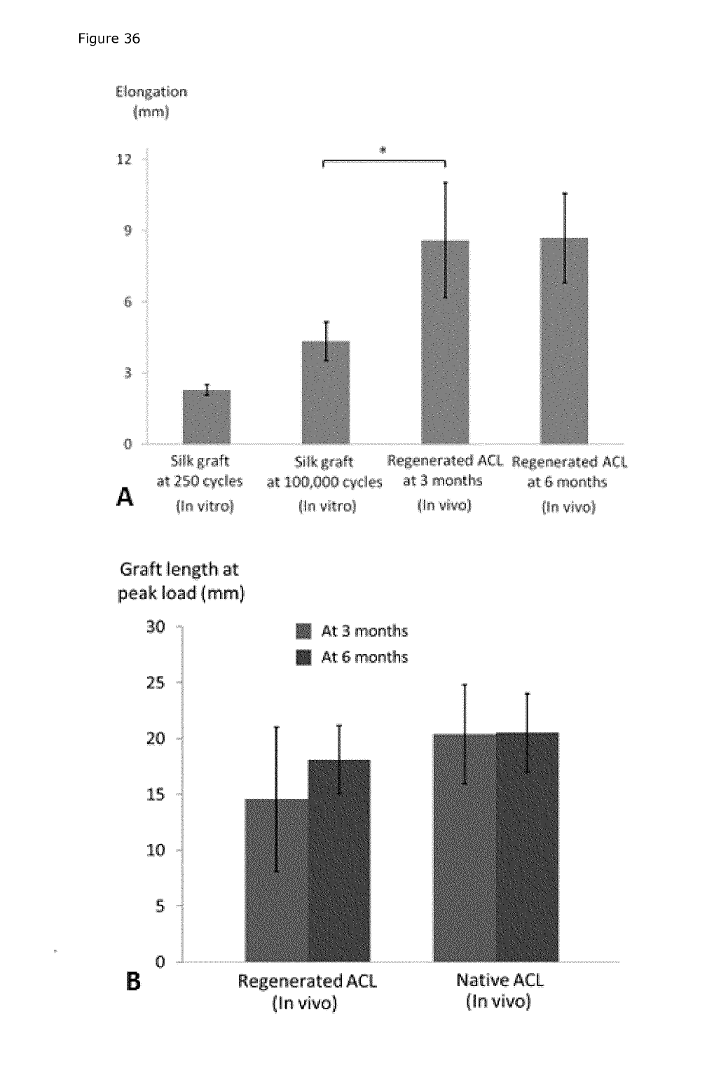

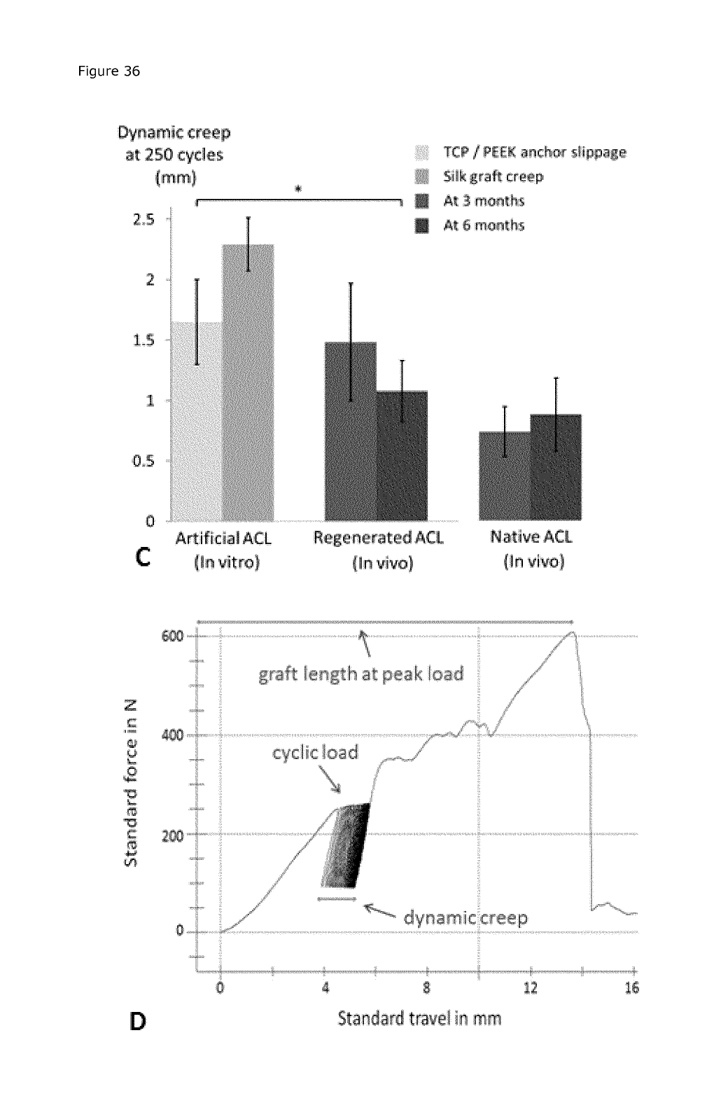

FIG. 36 shows comparison of mechanical properties of silk graft, TCP/PEEK anchor, regenerated ACL, native ACL at different time point. (p<0.05, A: Elongation; B: Graft length at peak load; C: Dynamic creep; D: Force displacement loading curve);

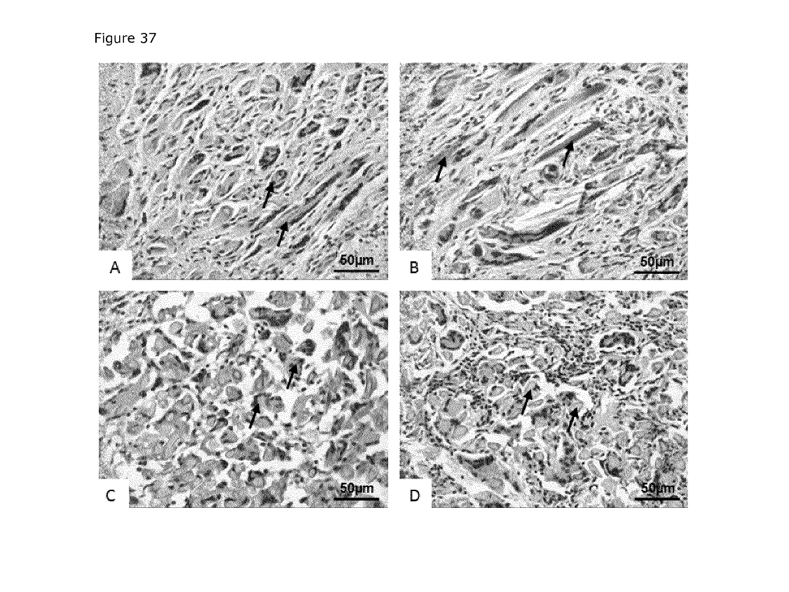

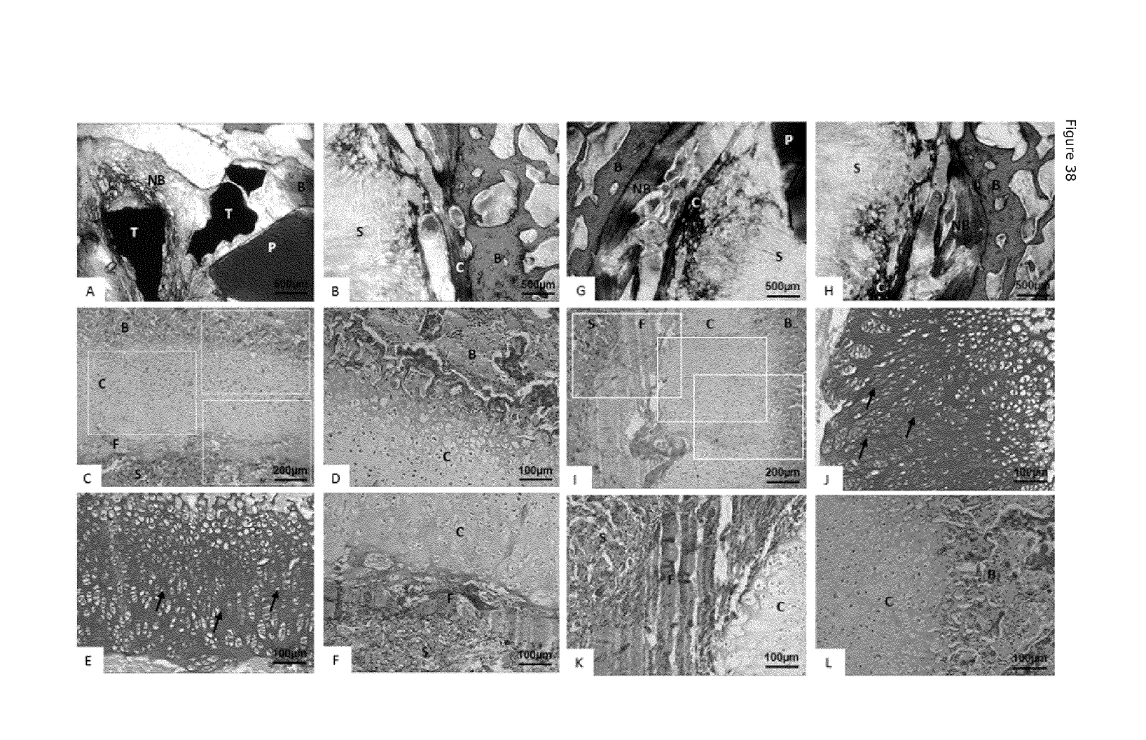

FIG. 37 shows hematoxylin and eosin stain of the silk graft with the regenerated fibro tissues at three months (A,C) and six months (B,D) time point (Black arrows point the silk fiber). A,B: Longitudinal section; C,D: Transverse section;

FIG. 38 shows histological images of silk graft to bone transitional zone in the femur tunnel. (A to F: at three months; G to L: at six months); (T: TCP; P: PEEK; B: bone; NB: newbone; C: fibrocartilage; F: fibrous tissue; S: silk); A,B,G,H: Goldner's trichrome stain; C,I: Hematoxylin and Eosin stain; D,F,K,L: Masson stain; E,J: Gomori stain;

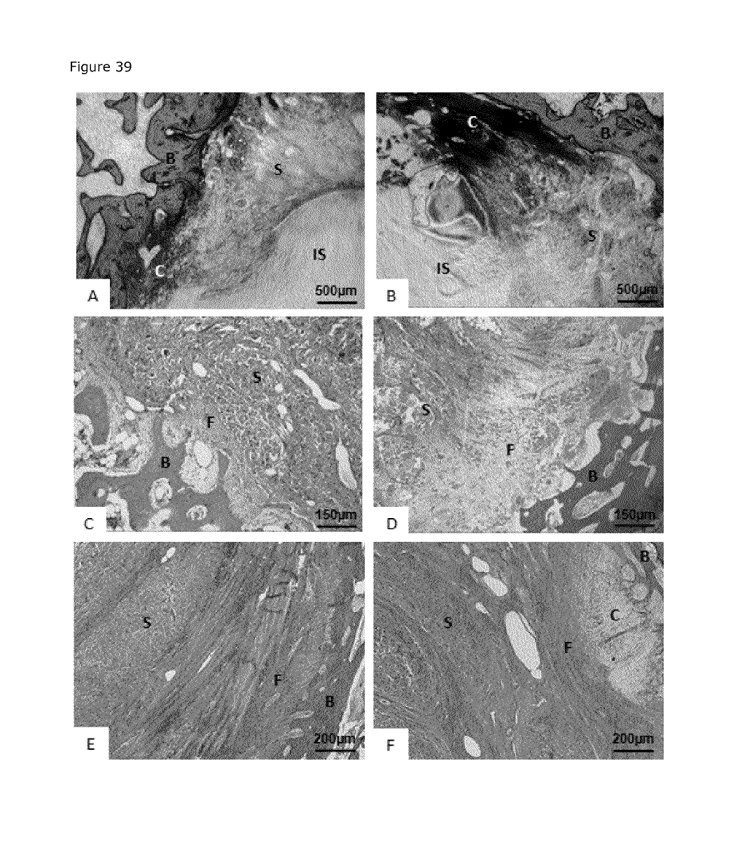

FIG. 39 shows histological images of silk graft to bone transitional zone in the tibia tunnel. (A,C: three months; B,D,E,F: six months); (IS: interference screw; B: bone; C: fibrocartilage; F: fibrous tissue; S: silk); A,B: Goldner's trichrome stain; C,D: Hematoxylin and Eosin stain; E,F: Masson stain;

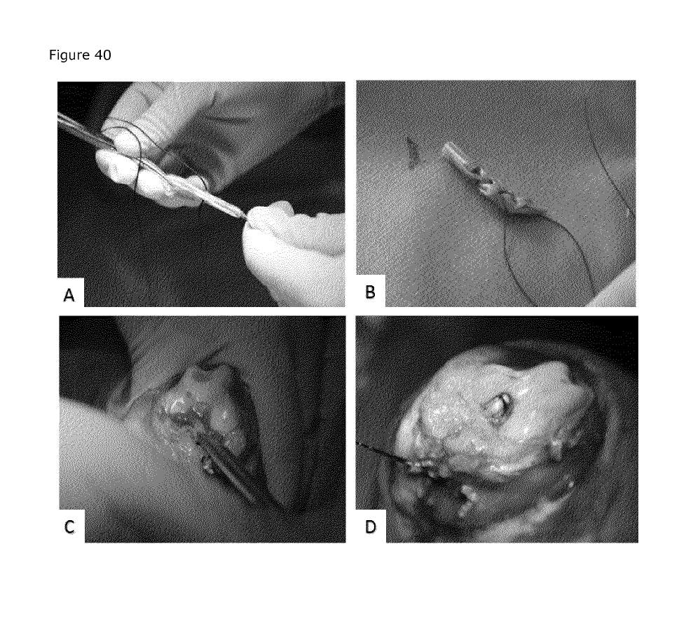

FIG. 40 shows canine CCL reconstructions with TCP/PEEK anchored tendon autograft; and

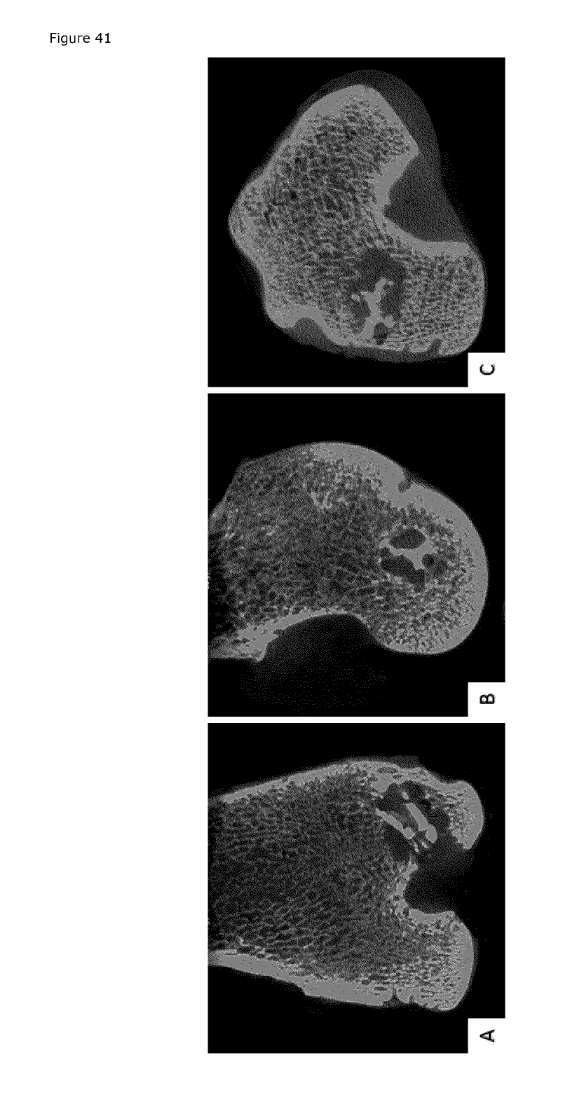

FIG. 41 shows CT images of the femoral tunnel with TCP/PEEK anchored tendon graft in canine model at three months' time point. (A: coronal view; B: sagittal view; C: transverse view);

FIG. 1 shows a device 1 according to the invention for fixation of a flexible element 10 to a particularly human bone 20, e.g. distal femur, in case the flexible element 10 is used for ACL reconstruction. The device 1 according to the invention comprises an insert 100 for holding the flexible element 10, which is particularly looped around the insert 100 as well as an anchor 200 into which said insert 100 is inserted. When inserted into a bore hole 2 of said bone 20, the anchor 200 contacts the walls of the bore hole 2 with its toothed outside 200a. At the same time the insert 100 is inserted such into the anchor 200 that also contact surfaces 112a, 113a, 122a, 123a of the insert 100 (cf. also FIGS. 6 and 7) contact the walls of the bore hole 2.

Preferably, the anchor 200 is made out of or comprises polyether ether ketone (PEEK) whereas the insert 100 preferably contains tricalcium phosphate (TCP). While the anchor 200 serves to provide adequate mechanical fixation in the beginning and thus good initial stability, the TCP insert 100 that holds the flexible element 10 is designed to promote bone cell ingrowth into the porous TCP scaffold, so that the flexible element 10, which may be a silk ACL scaffold or tendon autograft, see below, will be hold by the TCP/bone interface within the bore hole 2 of the bone 20. In the long-term, the TCP scaffold provided by insert 100 will be fully regenerated with the new born bone, and the flexible element 10 (e.g. silk ACL scaffold or tendon autograft) will be attached onto the native bone tissue firmly. The biological fixation will be finally achieved.

FIGS. 2 to 7 show the components of a device 1 according to the invention that is preferably used for a fixation of a synthetical flexible element 10 such as an ACL scaffold shown in FIGS. 12 and 13. As shown in FIGS. 2 to 5, the anchor 200 of the device 1 comprises a head part 201 having an annular shape and delimiting an opening 202 for passing through the flexible element 10 as shown in FIG. 1

The anchor 200 further comprises two legs 210, 220 protruding from the head part 201 along an insertion direction Z along which the anchor 200 and inserted insert 100 is inserted into the bore hole 2 with the legs 210, 200 of the anchor 200 ahead. The legs 210, 220 each comprise a concave inner surface 210a, 220a, which concave inner surfaces 210a, 220a face each other. Further, each leg 210, 220 comprises two lateral surfaces 210b, 220b, as indicated in FIGS. 4 and 5, coming off opposing edges 210c, 220c of the respective inner surface 210a, 220a. The lateral surfaces 210b, 220b are tilted by an angle W′ of 45° with respect to an extension plane spanned by said edges 210c of the respective leg 210, 220 (cf. FIG. 4).

According to FIGS. 6 and 7, the insert 100 comprises a first and a second wall region 101, 102 integrally connected by a connecting region 103, which comprises a concave surface 103a. The two wall regions 101, 102 and the connecting region 103 form a groove 104 or open channel 104 circulating around the connecting region 103 for receiving the flexible element 10, when the latter is laid around the connecting region 103 contacting the concave surface 103a of the connecting region 103 and the adjacent surfaces of the two opposing wall regions 101, 102.

The two wall regions 101, 102 each comprise a guiding recess 110, 120 extending along the insertion direction Z or longitudinal axis L of the insert 100 for guiding the insert 100 with respect to the anchor 200 upon insertion of the insert 100 into the anchor 200 counter to later insertion direction Z. Each guiding recess 110, 120 is delimited by a convex surface 110a, 120a of the respective wall region 101, 102, wherein the surfaces 110a, 120a face away from each other, and wherein each surface 110a, 120a is a section of a surface area of a cone, so that the surfaces 110a, 120a comprise a central radius R that decreases along the longitudinal axis L of the insert. This means that the insert 100 is correspondingly tapered in the region of the surfaces 110a, 120a. Further, each guiding recess 110, 120 is delimited by two opposing boundary regions 112, 113, 122, 123 extending along the longitudinal axis L of the insert 100. Each boundary region 112, 113, 122, 123 may comprise a wedge-like shape having an angle of W=45° particularly, as shown in FIG. 6.

Each boundary region 111, 112, 122, 123 of the insert 100 further comprises a contact surface 111a, 112a, 122a, 123a which is essentially flush with the outside 200a of the anchor 200 when the insert 100 is inserted into the anchor 200. These contact surfaces 111a, 112a, 122a, 123a serve for forming an interface between the TCP insert 100 and the walls of the bore hole 2, thus promoting bone cell ingrowth into the insert 100.

When inserting the insert 100 into the anchor 200 as shown in FIG. 3 the concave inner surfaces 210a, 220a of the legs 210, 220 of the anchor 200 slide on the convex surfaces 110a, 120a of the respective guiding recess 110, 120 of the insert 100 and press the legs 210, 220 away from each other, which allows for anchoring the anchor 200 in the bore hole 2. For this, the anchor 200 is inserted into the bore hole 2 when the insert 100 is not fully inserted into the anchor 200. Once the anchor 200 is in place, the insert 100 is pulled via the flexible element 10 attached to the insert 100 into its final position thereby pressing said legs 210, 220 away from each other so that the legs 210, 220 are pressed against the walls of the bore hole 2.

Further, when inserting the insert 100 into the anchor 200, the four lateral surfaces 210b, 220b of the legs 210, 220 slide along the boundary regions 111, 112, 113, 123 of the insert 100, thus prohibiting turning of the insert 100 with respect to the anchor 200. In this way the legs 210, 220 of the anchor 200 are guided in a form-fitting manner in the guiding recesses 110, 120 of the insert 100 upon insertion of the insert 100 into the anchor 200.

In other words, by means of the central Radius R (besides its spreading function) a primary guiding system is established which is supported by a secondary guiding system provided by the tilted lateral surfaces 210b, 220b (e.g. having said angle W′), which avoids turning of the insert 100 while implanting the device 1. As a second function, the secondary guidance system provides a contact zone (e.g. via contact surfaces 111a, 112a, 122a, 123a) between the TCP insert 100 and the bone 20, which is crucial for the osteoinduction or osteoconduction.

Further, FIGS. 8 to 11 show a further embodiment of a device 1 for fixation of a flexible element 10 to a bone 20, which is preferably used for natural flexible elements 10 such as ligament or tendon autografts. The device 1 has the same features as described above, but in contrast to the device 1 shown in FIGS. 2 to 7, the insert 100 has no tapered surfaces 110a, 120a. Further, the legs 210, 220 are relatively thinner, and the head part 201 does not comprise an annular shape, but two opposing cut-outs 203, 204 as shown in FIGS. 8 and 9, which receive the flexible element 10, so that the latter can be passed by the head part 201.

In case the insert 100 comprises no tapered regions (see above), the anchor 200 is pressed into the bore hole 2 with the insert 100 being fully inserted into the anchor 200.

Preferably, the afore-described anchors 200 are formed out of PEEK. PEEK anchors 200 can be fabricated with traditional machine tools. However, for the TCP insert 100, the geometry is quite complicated, which is not easy to produce by traditional machine tools. So we used an advanced manufacturing technique of combining rapid prototyping and gel-casting methods. The negative pattern of the TCP insert 100 was designed with a commercial Computer Aided Design (CAD) software (Pro-engineer). The molds were fabricated on a stereolithography apparatus (SPS 600B, xi′an jiaotong university, Xi′an, China) with a commercial epoxy resin (SL14120, Huntsman). The CAD data of the negative pattern was converted into STL data by Pro-engineer, imported into Rpdata software, and converted into an input file for stereolithography. The molds fabricated were then cleaned with isopropanol alcohol. TCP powders along with monomers (acrylamide, methylenebisacrylamide), and dispersant (sodium polymethacrylate) were mixed with deonized (DI) water to form a ceramic slurry. Table 1 shows an example of the amount of chemicals added to DI water to formulate a ceramic slurry used for forming an insert 100.

TABLE 1
Composition of slurry for scaffold fabrication

| | Component | Amount | |
|---|---|---|---|
| Solvent: | Deionized water | 35 | g |
| Ceramic powder: | Beta-tricalcium phosphate | 60 | g |
| Monomer: | Acrylamide | 4 | g |
| Cross linker: | Methylenebisacrylamide | 0.5 | g |
| Dispersant: | Sodium polymethacrylate | 0.6 | g |
| Initiator: | Ammonium persulphate | 0.2 | g |
| Catalyst: | N,N,N′N′-tetramethylethylenediamine | 0.1 | g |

The slurry prepared was deagglomerated by ultrasonic for 5 hours and subsequently deaired under vacuum until no further release of air bubbles from the sample. Catalyst (ammonium persulphate, (NH₄)₂S₂O₈) and initiator (N,N,N′N′-tetramethylethylenediamine) were added to the slurry to polymerize the monomers. The amount of which were controlled to allow a sufficient time for casting process. The TCP slurry was cast into the molds under vacuum to force the TCP powders to migrate into the interspaces of the paraffin spheres. The samples were dried at room temperature for 72 hours. After the drying, pyrolysis of the epoxy resin molds and paraffin spheres were conducted in air in an electric furnace with a heating rate of 5° C./h from room temperature to 340° C., holding 5 hours at 340° C. to ensure most paraffin spheres were burn out, and then sintered to 660° C. at a rate of 10° C./h, holding 5 hours at 660° C. to ensure most epoxy resin was burn out. After that the heating rate went up to 60° C./h till 1200° C., holding 5 hours at 1200° C., and then decreased to room temperature in 48 hours.

The mechanical property of porous TCP inserts or scaffold 100 varies with different porosities. The TCP inserts with different porosities have different elastic modules, and different failure stresses. To choose the proper porosity of porous TCP insert 100, a finite element analysis (FEA) was used to find out the stress and strain distribution on a TCP insert 100 while anchoring and pulling. Three different porosities, namely 40%, 60%, and 80%, were used in this study. It was found, that the maximum stress point is on the lower middle of the connecting region 103 of the TCP insert 100. For 60% porosity, under 1,000 N pullout force, the maximum stress on the TCP insert 100 is ˜1 GPa.

According to a preferred embodiment of the invention ACL scaffolds based on silk as shown in FIGS. 12 and 13 are used as flexible elements 10.

For producing such flexible elements 10 raw silk fibers (Bombyx mori) were obtained from Trudel Limited (Zurich, Switzerland). A special designed wiring machine was used to fabricate silk ACL scaffolds 10. For description purposes, the geometries of different hierarchical architectures were labeled as A(a)*B(b)*C(c)*D(d), where A, B, C, D represent the structural levels, which means number of fibers (A), bundles (B), yarns (C) and cords (D) in the final structure, while a, b, c, d is the twisting level, which means the lengths (mm) per turn on each of the hierarchical levels. After comparison and test with different structures, a wired silk scaffold structure was found to have the similar mechanical properties with human ACL. The structure parameter is defined as 6(0)*2(2)*144(10)*2(12), which means 6 fibers 303 in 1 bundle 302 without twist (0 means parallel), 2 bundles 302 in 1 yarn 301 with 2 mm per turn, 144 yarns 301 in 1 cord 300 with 10 mm per turn, 2 cords 300 in 1 ACL scaffold 10 with 12 mm per turn.

FIG. 13 shows an alternative embodiment of a flexible element 10 in the form of a braided ACL scaffold. Here, the structure parameter is defined as 6(0)*2(2)*96(10)*3(12), which means 6 fibers 303 in 1 bundle 302 without twist (0 means parallel), 2 bundles 302 in 1 yarn 301 with 2 mm per turn, 96 yarns 301 in 1 cord 300 with 10 mm per turn, 3 braided cords 300 in 1 ACL scaffold 10 with 12 mm per turn.

The flexible elements in the form of silk ACL scaffolds 10 depicted in FIGS. 12 and 13 were produced with raw silk yarns. The hyper-antigenic protein sericin was removed by immersing the scaffolds 10 into an aqueous solution of 0.5 wt % Na₂CO₃ at 90° C.-95° C., 300 RPM in a magnetic stirrer (Basic C, IKA-WERKE, Germany) for 90 minutes, then rinsing with running distilled water for 15 minutes, and air dried at 60° C. These procedures were repeated three times, then the sericin was thoroughly extracted. Scanning electron microscopy (FEG-SEM, Zeiss LEO Gemini 1530, Germany) with an in-lens detector was used to view the surface of the silk fiber to evaluate the extracting protocol. Prior to imaging, the scaffolds 10 were coated with platinum in order to allow imaging at better resolutions. The SEM image of surface of the original silk fibers is shown in FIG. 31 (left panel), and the image of sericin-extracted fibers is shown in FIG. 31 (second panel from the left). To evaluate cell adherence on the silk ACL scaffold, human foreskin fibroblasts (HFFs) prelabeled with Calcein AM (i.e., the acetomethoxy derivate of calcein) were seeded on the scaffold, and were imaged on an upright Leica microscope with the appropriate excitation and emission filters. FIG. 31 (third panel from the left) shows the fluorescein microscope images of silk scaffold with HFF cells seeded on the silk scaffold 30 minutes later. FIG. 31 (fourth panel from the left) shows the HFF cells seeded on the silk scaffold 24 hours later. We can see the HFF cells are clearly attached and aligned with silk fibers very well after 24 hours.

For biomechanical testing of the silk-based flexible elements 10 in vitro pull to failure tests and low-cycle-loading tests were performed on a universal material testing machine (Zwick 1456, Zwick GmbH, Ulm, Germany), wherein a 20 kN force sensor (Gassmann Theiss, Bickenbach, Germany) was used. A special fixation clamp was developed. The distance between the clamps was 30±1 mm to simulate the normal ACL length [48, 49]. For the initial pull to failure tests a pre-conditioned loading of 5 N was applied to the flexible element (e.g. scaffold) 10, and afterwards a displacement-controlled loading of 0.5 mm/second was applied to the scaffold 10. For the low-cycle loading test, after applying a pre-conditional loading of 5 N to the scaffold 10, a force controlled cyclic loading from 100 N to 250 N over 250 cycles, representing the loads of normal walking [50], were applied with a loading speed of 0.5 mm/second.

To simulate long term loading of flexible elements (e.g. ACL scaffolds) 10, a specialized bioreactor 400 shown in FIG. 14 was employed. A stepper motor 401 (e.g. NA23C60, Zaber Technologies Inc, Canada) was used to apply cyclic load, and a 1 kN load cell (e.g. KMM20, Inelta Sensorsystems, Germany) 404 was used to acquire the force. For holding the flexible element to be tested, said bioreactor 400 comprises two clamps 402 and a chamber 403, particularly in the form of a tube made of Polysulfon (PSU1000, Quadrant AG, Switzerland) surrounding the scaffold 10 to be tested as well as the clamps 402. The bioreactors 400 were fixed in an incubator (C150, Binder, Germany), and controlled by a special developed program with LabVIEW (9.x).

The length of the tested silk scaffolds 10 between the clamps was 28±3 mm. The chamber 403 was filled with PBS and covered with an aluminum foil cap. The temperature in the incubator was 37° C. The humidity was 100%, and the CO₂ concentration was 5%. After applying a pre-conditional loading of 5 N, the high-cycle loading was applied with a strain control at 1 Hz frequency of 3% strain over 100,000 cycles with an interval rest of 30 seconds between every 250 cycles.

Mechanical properties of silk yarns with different conditions had been tested. FIG. 22 shows the ultimate tensile strength (UTS) and FIG. 23 the linear stiffness of silk yarns in three conditions, respectively: native silk yarn before sericin extraction, silk yarn after sericin extracted in dry condition, and silk yarn after sericin extracted in wet condition, which means the test samples were treated with PBS for 30 minutes before test. The geometry of silk yarn is 6(0)*2(2) as described previously. The lengths of each sample is 30 mm, and the diameter is 0.24 mm of native silk yarn, 0.17 mm of sericin extracted (dry), and 0.14 mm of sericin extracted (wet). The detailed data is listed in Table 2. The UTS of silk yarns decreased quite remarkably after sericin extraction, from 9.42±0.33 N of native silk yarn to 7.34±0.35 N of sericin extracted (dry) and 6.00±0.33 N of sericin extracted (wet), respectively, shown in FIG. 22. The stiffness of silk yarns decreased as well after sericin extraction, from 1.97±0.07 N/mm of native silk yarn to 1.37±0.17 N/mm of sericin extracted (dry) and 1.03±0.23 N/mm of sericin extracted (wet), respectively. The stiffness of silk yarns reduced significantly (p<0.01) in wet condition, shown in FIG. 23. The failure elongation of silk yarns also decreased after sericin extraction, from 9.8±0.33 mm of native silk yarn, to 8.14±0.30 mm of sericin extracted (dry) and 6.94±0.40 mm of sericin extracted (wet) respectively, shown in Table 2.

TABLE 2
Mechanical properties of silk yarn in three conditions

| | | | | | | | UTS ± | Stiffness ± | Elongation ± | | |
|---|---|---|---|---|---|---|---|---|---|---|---|
| | | | Lengths | Diameter | | Total | stdev | stdev | stdev | UTS | Stiffness |
| Architecture | Geometry | Condition | (mm) | (mm) | Cycles | fibers | (N) | (N/mm) | (mm) | per fiber | per fiber |
| Silk yarn | 6(0)*2(2) | Unextracted, | 30 | 0.24 | 0 | 12 | 9.42 ± 0.33 | 1.97 ± 0.07 | 9.08 ± 0.33 | 0.79 | 0.16 |
| | | Dry | | | | | | | | | |
| Silk yarn | 6(0)*2(2) | Extracted, | 30 | 0.17 | 0 | 12 | 7.34 ± 0.35 | 1.37 ± 0.17 | 8.14 ± 0.30 | 0.61 | 0.11 |
| | | Dry | | | | | | | | | |
| Silk yarn | 6(0)*2(2) | Extracted, | 30 | 0.14 | 0 | 12 | 6.00 ± 0.33 | 1.03 ± 0.23 | 6.94 ± 0.40 | 0.50 | 0.09 |
| | | Wet | | | | | | | | | |

Pull to failure tests had been performed on silk ACL scaffolds 10 with different architectures. All the samples were sericin-extracted, tested in dry and wet conditions, respectively. The architectures of silk ACL scaffolds are: parallel 6(0)*2(2)*288(10)*1(0), wired 6(0)*2(2)*144(10)*2(12), and braided 6(0)*2(2)*96(10)*3(12), as described previously. FIG. 24 shows the UTS and FIG. 25 the linear stiffness of silk ACL scaffolds 10 of three architectures. It is obvious that the silk ACL scaffold 10 with parallel architecture has a lower UTS and higher stiffness, which is much farther from the value of human ACL taken from Woo et al [51]. The UTS of wired and braided architectures decreased significantly (P<0.01) from around 1900 N in dry condition to around 1500 N in wet condition, shown in FIG. 24. Although the value is lower than that of human ACL, it is still acceptable in ACL tissue engineering, since the earlier report shows that the UTS of human ACL varies according to the ages, up to 1730 N in people aged 16-26 years, but much less in people aged 48-86 years, with a mean average for approximately 734 N [52]. The stiffness of wired and braided architectures also decreased significantly (P<0.01) from around 550 N/mm in dry condition to around 250 N/mm in wet condition, which is quite close to the value of human ACL, shown in FIG. 25. To find out the effect of sterilization procedures on mechanical properties of silk ACL scaffolds 10, three samples of wired silk ACL scaffolds after sterilization had been tested. The UTS, linear stiffness, and failure elongation were 1444±102 N, 251±39 N/mm, and 3.93±0.36 mm respectively. The detailed data is listed in Table 3.

TABLE 3
Mechanical properties of silk scaffolds with three architectures in dry and wet conditions

| | | | | | | | UTS ± | Stiffness ± | Elongation ± | | |
|---|---|---|---|---|---|---|---|---|---|---|---|
| | | | Lengths | Diameter | | Total | stdev | stdev | stdev | UTS | Stiffness |
| Architecture | Geometry | Condition | (mm) | (mm) | Cycles | fibers | (N) | (N/mm) | (mm) | per fiber | per fiber |
| Parallel | 6(0)*2(2)*28 | Extracted, | 30 ± 1 | ~6.7 | 0 | 3456 | 1589 ± 98 | 1258 ± 57 | — | 0.46 | 0.36 |
| | 8(10)*1(0) | Dry | | | | | | | | | |
| Parallel | 6(0)*2(2)*28 | Extracted, | 30 ± 1 | ~6.1 | 0 | 3456 | 1452 ± 106 | 822 ± 60 | — | 0.42 | 0.24 |
| | 8(10)*1(0) | Wet | | | | | | | | | |
| Wired | 6(0)*2(2)*14 | Extracted, | 30 ± 1 | ~6.5 | 0 | 3456 | 1910 ± 128 | 567 ± 40 | — | 0.55 | 0.16 |
| | 4(10)*2(12) | Dry | | | | | | | | | |
| Wired | 6(0)*2(2)*14 | Extracted, | 30 ± 1 | ~6.2 | 0 | 3456 | 1543 ± 85 | 289 ± 21 | 3.94 ± 0.25 | 0.45 | 0.08 |
| | 4(10)*2(12) | Wet | | | | | | | | | |
| Wired | 6(0)*2(2)*14 | Extracted, | 30 ± 1 | ~6.2 | 0 | 3456 | 1444 ± 102 | 251 ± 39 | 3.93 ± 0.36 | 0.42 | 0.07 |
| | 4(10)*2(12) | Wet | | | | | | | | | |
| | | Sterilized | | | | | | | | | |
| Braided | 6(0)*2(2)*96 | Extracted, | 30 ± 1 | ~6.6 | 0 | 3456 | 1890 ± 62 | 546 ± 41 | — | 0.55 | 0.16 |
| | (10)*3(12) | Dry | | | | | | | | | |
| Braided | 6(0)*2(2)*96 | Extracted, | 30 ± 1 | ~6.1 | 0 | 3456 | 1599 ± 65 | 242 ± 26 | 4.75 ± 0.26 | 0.46 | 0.07 |
| | (10)*3(12) | Wet | | | | | | | | | |

Cyclic load tests had been performed on silk ACL scaffolds 10 with wired and braided architectures (cf. also FIGS. 12 and 13). The UTS and linear stiffness of scaffolds were compared under the following loading conditions: without loading, low cycle loading and high cycle loading. Cells were seeded on the scaffolds 10 to find out the effect of cell on the mechanical behavior of silk ACL scaffold 10 under different loading conditions. For the samples without cyclic loading, the UTS decreased slightly after immersed into the PBS solution for 7 days, from 1543±85 N to 1362±20 N for wired architecture, and from 1599±65 N to 1391±12 N for braided architecture. After cyclic loading, the UTS reduced significantly, to ˜900 N (wired) and ˜800 N (braided) after 250 cycles, to ˜500 N (wired) and ˜400 N (braided) after 100,000 cycles, shown in FIG. 26. The linear stiffness of samples without cyclic loading also decreased slightly after immersed into PBS solution for 7 days, from 289±21 N/mm to 236±23 N/mm for wired architecture, and from 242±26 N/mm to 207±31 N/mm for braided architecture. After cyclic loading, the linear stiffness increased significantly, to 428±32 N/mm (wired) and 518±66 N/mm (braided) after 250 cycles, to 490±14 N/mm (wired) and 553±38 N/mm (braided) after 100,000 cycles, shown in FIG. 27. There is no significant difference on mechanical properties (P>0.05) for wired architectures between the silk ACL scaffold 10 with cells and without cells under high cyclic loading. The detailed data is listed in Table 4.

TABLE 4
Mechanical properties of wired and braided silk scaffolds with three architectures under different conditions.

| | | | | | | | UTS ± | Stiffness ± | Elongation ± | | |
|---|---|---|---|---|---|---|---|---|---|---|---|
| | | | Lengths | Diameter | | Total | stdev | stdev | stdev | UTS | Stiffness |
| Architecture | Geometry | Condition | (mm) | (mm) | Cycles | fibers | (N) | (N/mm) | (mm) | per fiber | per fiber |
| Wired | 6(0)*2(2)*14 | Extracted, | 30 ± 1 | ~6.2 | 250 | 3456 | 913 ± 123 | 428 ± 32 | 5.51 ± 0.32 | 0.26 | 0.12 |
| | 4(10)*2(12) | Wet | | | | | | | | | |
| Braided | 6(0)*2(2)*96 | Extracted, | 30 ± 1 | ~6.1 | 250 | 3456 | 787 ± 69 | 518 ± 66 | 5.56 ± 0.36 | 0.23 | 0.15 |
| | (10)*3(12) | Wet | | | | | | | | | |
| Wired | 6(0)*2(2)*14 | Extracted, | 28 ± 3 | ~6.2 | 100,000 | 3456 | 520 ± 76 | 490 ± 14 | 4.34 ± 0.81 | 0.15 | 0.14 |
| | 4(10)*2(12) | Wet | | | | | | | | | |
| Braided | 6(0)*2(2)*96 | Extracted, | 28 ± 3 | ~6.1 | 100,000 | 3456 | 401 ± 76 | 553 ± 38 | 4.25 ± 0.46 | 0.12 | 0.16 |
| | (10)*3(12) | Wet | | | | | | | | | |
| Wired | 6(0)*2(2)*14 | Static cell | 30 ± 1 | ~6.2 | 0 | 3456 | 1489 ± 82 | 274 ± 22 | — | 0.43 | 0.11 |
| | 4(10)*2(12) | culture | | | | | | | | | |
| | | 24 hours | | | | | | | | | |
| Wired | 6(0)*2(2)*14 | Static cell | 30 ± 1 | ~6.2 | 0 | 3456 | 1362 ± 20 | 236 ± 23 | — | 0.39 | 0.07 |
| | 4(10)*2(12) | culture | | | | | | | | | |
| | | 7 days | | | | | | | | | |
| Braided | 6(0)*2(2)*96 | Static cell | 30 ± 1 | ~6.1 | 0 | 3456 | 1572 ± 89 | 246 ± 28 | — | 0.45 | 0.71 |
| | (10)*3(12) | culture | | | | | | | | | |
| | | 24 hours | | | | | | | | | |
| Braided | 6(0)*2(2)*96 | Static cell | 30 ± 1 | ~6.1 | 0 | 3456 | 1391 ± 12 | 207 ± 31 | — | 0.40 | 0.06 |
| | (10)*3(12) | culture | | | | | | | | | |
| | | 7 days | | | | | | | | | |
| Wired | 6(0)*2(2)*14 | Dynamic | 28 ± 3 | ~6.2 | 100,000 | 3456 | 502 ± 41 | 489 ± 12 | — | 0.15 | 0.14 |
| | 4(10)*2(12) | cell | | | | | | | | | |
| | | culture 48 | | | | | | | | | |
| | | hours | | | | | | | | | |

The linear stiffness and elongation of silk ACL scaffold 10 under high cyclic loading were recorded. The linear stiffness of silk ACL scaffold 10 increased sharply from 289±21 N/mm (wired) and 242±26 N/mm (braided) at 0 cycle, to 428±32 N/mm (wired) and 518±66 N/mm (braided) at 250 cycles, then increased slightly to 496±13 N/mm (wired) and 556±37 N/mm (braided) at 20,000 cycles, and remain stable at ˜500 N/mm (wired) and 550 N/mm (braided) until 100,000 cycles. The elongation of silk ACL scaffold 10 increased sharply from 0 at the beginning to 2.3±0.2 mm (wired) and 1.2±0.1 mm (braided) at 250 cycles, and increased gradually to 3.6±0.4 mm (wired) and 3.0±0.3 N/mm (braided) at 10,000 cycles, then increased slightly to 4.3±0.8 mm (wired) and 4.3±0.5 mm (braided) at 100,000 cycles, shown in FIG. 28.

The PEEK anchors 200 were tested on a universal material testing machine (Zwick 1456, Zwick GmbH, Ulm, Germany), the testing protocol was the same as previously described. The distance between the clamps was 30±1 mm to simulate the normal ACL length [48,49]. For the tests a pre-conditioned loading of 5 N was applied to the anchor 200, and afterwards a displacement-controlled loading of 0.5 mm/second from 100 to 250 N over 250 cycles was applied to the anchors 200, representing the loads of normal walking [50], and then a pullout to get the ultimate tensile strength. Three types of anchors, V0, V1, and V2, were tested. V0 denotes an insert having parallel wall regions 101, 102 (i.e. non-tapered insert 100), which have no spreading effect on the anchor 200. The V1 and the V2 system have a small wedge and a bigger wedge respectively (cf. FIG. 3). Table 5 shows the failure and survival samples out of the whole test. We can find out from the table that the anchors 200 with spreading effect as shown in FIG. 3 have a better survival rate.

TABLE 5

| System | n total | n useful | n failure |
|---|---|---|---|
| V0 | 10 | 7 | 3 |
| V1 | 7 | 7 | 0 |
| V2 | 9 | 9 | 0 |

Regarding the slippage in pig bone, the results of the V1/V2-system are quite good, shown in FIG. 29. The mean values of these two systems are around 0.7 mm, which is an improvement of around 56% compared with the V0-system. As it was intended to keep the slippage below a value of 1.5 mm, the system can in this sense be regarded as successful.

The untimate tensile strengths (UTS) are shown in FIG. 30. As can be seen from FIG. 30, the V2-System is comparable to an 8/28 Interference screw (IS). In mean, the IS is slightly higher than the V2 (715N to 684N). However, the median of the V2 is slightly higher than the median of the IS (698N to 694N). A T-Test comparing the V2 with the IS-Groups shows no significant difference of the mean values (P=0.695) between these groups. In this sense, the V2 can be regarded as an equivalent system to the IS regarding the ultimate tensile strength.

In order to further verify the concept of this design, a pilot animal study (in vivo) had been carried out on 2 porcines for 3 months from 9 Jan. 2012 to 8 Apr. 2012. The porcine was ˜1.5 months old and ˜50 kg in weight, the growth rate is ˜2 kg per week. The silk ACL scaffold 10 with TCP insert 100 and PEEK anchor 200 for this animal study were prepared under strict GMP standard.

The surgical processes can be inferred from FIG. 15. A first approach is minimally invasive, similar to ACL repair surgery currently used in clinics. First, make a small lateral incision to put endoscope into the knee joint. Then, a trans-tibial bone tunnel 2d of 7 mm diameter is drilled, as well as a bore hole 2 of 20 mm length in the femoral distal. Then, bend the knee, and make a medial incision. Enlarge the bore hole 2 to 9 mm diameter through the medial incision. Afterwards, the insert 1 is inserted and anchored using the first tool 40, through the medial incision. Then, the free end of the flexible element (e.g. ACL scaffold) 10 is pulled through the tibia tunnel 2d. The silk scaffold 10 is pulled tight, tension is adjusted by the surgeon, and fixed with a standard interference screw (φ6×19 mm).

The results of the pilot animal study are quite promising. FIG. 32 shows the partly regenerated ligament tissue after euthanasia at 3 months. We can see clearly that fibro-tissue regenerated along with the silk fiber (flexible element) 10. From the micro-CT image, shown in FIG. 33, we can see the newborn bone formed and the fibro-tissue attached on the newborn bone and TCP insert 100.

For a final assessment on in vivo behavior, a second animal experiment was performed with 14 healthy adult male pigs (Chinese tri-hybrid pig: Xianyang breed) aged around four months and weighing 55.2±3.7 kg (mean±SD) at time of surgery. ACL reconstructions were performed on the left knee. The animals were divided into two study groups with 10 animals planned for sacrifice at a three-month time point, and 4 animals at a six-month time point. Within the three-month group, 7 of 10 animals were used for biomechanical tests, the remaining 3, plus 1 from the biomechanical test samples, (4 animals) were used for histological observation. From the six month group, 3 of 4 animals were used for biomechanical tests, with the remaining specimen combined with one specimen from the 3 biomechanical test samples (i.e. 2 animals) allocated for histological analysis.

An open surgical procedure for ACL reconstruction was used as previously described. Analgesics (100 mg pethidine) were given to each animal twice a day for three days following the surgery. In order to prevent infection, antibiotics (Penicillin of 800′000 U) were given to each animal twice a day until five days after operation. Disinfection solution (0.25% didecyl dimethyl ammonium bromide) was sprayed on the animals and bedding biweekly until the end of the experiment. All pigs were randomly assigned housing in one of three pens (5×8 m), and allowed unrestricted daily activity in their pen. Activity level and degree of lameness were monitored. As planned, ten pigs were euthanized by lethal injection of thiamylal sodium at a postoperative time point of three months. The remaining four pigs were euthanized at six months. After euthanization both knees were dissected. The samples used for biomechanical test (7 of 10 at three months, 3 of 4 at six months) were immediately stored at −20° C. The remaining samples used for histological observation were cut into small specimens and immediately fixed in a 10% buffered formalin solution. Radiological observation using standard c-arm device was performed on three pigs on the first postoperative day. At each euthanization time point, three additional knees were imaged for qualitative evaluation of TCP degradation and a rough characterization of new bone formation in the femur tunnel.

After ligament reconstruction all animals were standing upon three legs by the third postoperative day. All animals were walking on four legs with a detectable degree of lameness within 5 to 7 days postoperatively. Activity levels increased gradually after one week, until resumption of normal activity and no discernable lameness by the second postoperative week. At time of euthanization no animal exhibited graft failure or apparent degenerative changes in surrounding tissues of the knee (articular cartilage, menisci, other ligaments). Analysis of blood chemistry indicated no systemic markers of inflammation.

Lateral X-Ray images of the knee joint at three time points indicated progressive resorption of TCP (FIG. 34). The edge of the bone tunnel was clearly visible in the postoperative X-ray images, as was the TCP within the tunnel. At three months, an observable zone with a gradient of grey scales from TCP to the bone tunnel was present, demarcating regions of new bone formation. At six months, observable TCP regions were much smaller, but still present. The grey scale intensity of the bone tunnel was higher at six months compared to that at three months, qualitatively indicating increased presence of new bone and increased bone volume.

The length of silk graft at implantation was 33.6±4.2 mm (n=14). The length of the regenerated ACL was 42.2±3.4 mm at three months (n=7), and 43.3±2.9 mm at six months (n=3). The length of the native ACL was 37.4±3.2 mm at three months (n=7), and 37.3±2.1 mm at six months (n=3). Comparison of graft length to the contralateral (native) ligament revealed these differences to be non-significant (FIG. 35A). The cross section area of silk graft at implantation was 30.2±2.3 mm2 (n=14). The cross section area of the regenerated ACL was 57.5±8.1 mm2 at three months (n=7), and 84.6±11.5 mm2 at six months (n=3). The cross section area of the native ACL was 23.6±4.8 mm2 at three months (n=7), and 30.3±4.4 mm2 at six months (n=3). The comparison of the graft cross section areas indicated significant differences between area at time of implantation and then at three months (p<0.01), with a further significant increase between three months and six months (p=0.016; FIG. 35B).

Two regenerated ACL specimens sacrificed at 3 months failed prior to onset of cyclic loading (151 N and 184 N). Although the loading mode of these two failed samples were different with other samples, we also included UTS statistical analysis, but excluded for stiffness analysis. The UTS of native ACL was 1384±181 N at three months (n=7), and increased but not significantly (p=0.14) to 1749±284 N at six months (n=3), similar to reports in the literature. The UTS of the regenerated ACL was 311±103 N at three months (n=7), and increased significantly (p<0.01) to 566±29 N at six months (n=3) (FIG. 35C). All failures occurred in the midsubstance of the regenerated ACL—with no pullout failures observed at either the femoral tunnel or tibial tunnel. The stiffness was calculated as the slope of the force-displacement curve between 100 N and 250 N of the 250th cycle. The stiffness of native ACL was 192±22 N/mm at three months (n=5), and increased significantly (p<0.01) to 259±15 N/mm at six months (n=3). The stiffness of regenerated ACL was 148±19 N at three months (n=5), and increased significantly (p=0.035) to 183±10 N at six months (n=3) (FIG. 35D)

Compared to the graft length at time of implantation, there was a significant increase (p=0.04) in length of the regenerated ACL after three months in vivo—an increase in length that was larger than elongation observed after 100′000 cycles of in vitro testing (FIG. 36A). This parameter reflects any slippage of the anchor or interference screw, and creep in the graft. The graft length at peak load for the regenerated ACL was 14.6±6.5 mm at three months (n=7), and increased but not significantly (p=0.27) to 18.1±3.0 mm at six months (n=3), which was ˜10% lower than the value of native ACL (˜20 mm) (FIG. 36B). The dynamic creep of native ACL was 0.74±0.21 mm at three months (n=5), and 0.88±0.30 mm at six months (n=3). The dynamic creep of regenerated ACL was 1.48±0.49 mm at three months (n=5), and decreased but not significantly (p=0.145) to 1.07±0.25 mm at six months (n=3). There was a significant decrease (p=0.046) in dynamic creep comparing the TCP/PEEK anchor and the regenerated ACL at 6 months against in vitro data and the regenerated ACL at three months (FIG. 36C). From a functional standpoint, this efficacy study focused on the mechanical strength and stiffness of the regenerated ACL. The UTS of the regenerated ACL increased by ˜82% (FIG. 35C) from three months to six months. Although the absolute strength of the graft was still far from that of the native ACL, these values fall safely below typical maximal ACL loads associated with normal daily activity (˜250 N). The UTS values we recorded at 3 months compare favorably with other ACL reconstruction studies using porcine models with sacrifice after three months, although UTS values we recorded at 6 months were approximately 40% lower than another study with a similar time course. Also as in previous studies, failures almost nearly occurred in the midsubstance of the reconstructed ACL, with no incidence of tunnel pullout failure. It should be noted that graft elongation at failure typically exceeded 15 mm (FIG. 36B), a distance at which recruitment of other stabilizing structures (muscles, other ligaments) would reasonably be expected to prevent graft failure. Graft slippage and elongation also play a critical role in functional performance, as these aspects are closely related to loss of graft tension and relative joint laxity. The elongation of the regenerated ACL compared to graft length at implantation was ˜8.6 mm for both three months and six months (FIG. 36A), although interpreting these values is difficult in view of the fact that the animals were growing over the course of the experiment. More conclusively, elongation of silk graft during cyclic load (dynamic creep) of the regenerated ACL decreased by ˜38% from three months to six months, indicating that the graft become less viscoelastic in this timeframe. Still, because any measure of graft elongation includes effects from both the femoral side (silk/TCP/PEEK) and the tibial side (silk-IS), it was not possible to assess the relative contribution of either side to the overall function. Nonetheless when compared to in vitro biomechanical test data, dynamic creep of the regenerated ACL was ˜35% lower after 6 months than the original graft (FIG. 36C), clearly indicating that the implant became more elastic (less viscoelastic) over the course of healing—and was comparable to the native ACL.

Hematoxylin and eosin staining of longitudinal sections (along the axis of the tunnel) and transverse sections (perpendicular to the tunnel) indicated substantial fibrous tissue formation surrounding the silk fibers, with slightly increased presence at six months (FIG. 37). Silk-based scaffolds have been increasingly investigated as a potential graft material for tendon and ligament regeneration. This owes in part to the advantageous biological properties of silk as well as robust biomechanical strength in the short- and middle-term. After three months of postoperative healing, we observed that the silk scaffolds remained largely intact but were intermixed with regenerated fibrous tissue, the cells of which were well aligned with and often attached to the silk fibers (FIG. 37A,C). After six months the regenerated fibrous tissue that was intermixed with silk fibers appeared to be increased, although not substantially (FIG. 37B,D), with the majority of newly generated fibrous tissues forming around the silk graft core. The silk graft remained around 70% intact even at six months, reflecting the characteristically slow degradation of silk, which enables the biomaterial scaffold to continue supporting functional demands of the ligament until the host tissues eventually overtake these loads. These findings are consistent with extensive studies by others using silk grafts for ACL reconstruction. The fibrous tissues covered around the silk graft were disorganized and can be regarded as some kind of scar tissue. There were plenty of blood vessels in the scar tissue (pink color in FIG. 34E, 34F), which made it grown thicker over the regenerating process. The cross sectional area of regenerated ACL at six months was ˜47% larger than that at three months (FIG. 35B), which was mainly attributed by the growth of scar tissue. This scar tissue blocked the interstitial fluid to go deeper into the silk graft, which is the essential factor for silk degradation process. This is the reason that the degradation speed of silk graft was slower over the regenerating process.

Goldner's trichrome stain was adopted to observe the regenerated tissue in the bone tunnel. The TCP could still be located at three months, with regenerated new bone tissue observed to surround the TCP (FIG. 38A). New bone tissue was increasingly present at six months, with fibrocartilage observed to lie between silk fibers and the new bone tissue (FIG. 38G). The silk to bone transitional area was characterized with Hematoxylin and Eosin stain in terms of silk, fibrous tissue, fibrocartilage, and bone (FIG. 38C at three months and FIG. 38I at six months). The regenerated fibrous tissue layer characterized using Masson stain at six months (FIG. 38K) was nearly twice as thick as that at three months (FIG. 38F), reflecting the regeneration of fibrous tissue surrounding the silk graft. The connection of fibrous tissue to bone through a fibrocartilage zone, and Gomori staining revealed interdigitated (Sharpey's) fibers in fibrocartilage zones. Numerous such fibers could be seen both at three months (FIG. 38E) and six months (FIG. 38J).

At regions of contact between the silk, interference screw (IS), and bone in the tibial tunnel, cartilaginous tissue was observed at the silk-IS-bone interface at three months (FIG. 39A). This cartilagenous layer at the silk-IS-bone corner was even more pronounced at six months (FIG. 39B). However, at the interface of silk to bone the transition was characterized by the presence of silk, fibrous tissue, and bone tissue only, with no cartilaginous layer observable at three months (FIG. 39C) or six months (FIG. 39D,E). There were only a few cases showing a thin, non-continuous layer of fibrocartilage at six months (FIG. 39F). Comparison between the tibial and femoral tunnels revealed a relative absence of new bone formation in the tibial tunnel, with a corresponding lack of a cartilagenous silk to bone transition in the tibia tunnel.

The present study differentiates itself from previous studies, in its use of a porous TCP scaffold (mimicking a bone block) combined with a PEEK anchor. From histological observation, we found that the porous TCP scaffold substantially increased silk graft to bone attachment. There was a clear tendency toward new bone formation in the femur tunnel in contrast to the tibial tunnel which lacked presence of TCP (FIG. 38). At three months the TCP scaffold could still be seen clearly, while at six months considerable less TCP material could be identified, comparable to degradation rates reported in the literature. Over the course of TCP remodeling, the enlaced silk graft was apparently incorporated within the tunnel leading to apparently accelerated biological fixation by three months and robust incorporation to the tunnel by six months. In contrast, there was little new bone tissue formation observed in the tibial tunnel, particularly at the margins of the silk graft pressed against one side of the tunnel by the interference screw. Lacking a histological transition from the silk to the host bone, it would appear that silk graft fixation would remain dependent on mechanical purchase of the screw (FIG. 39A,B) and could thus remain susceptible to subsequent loosening.

In femur tunnel we conclude that the presence of TCP provoked formation of tissue transitions from silk, into regenerated fibrous tissue, into regenerated fibrocartilage, and eventually into bone (FIG. 38C,I). These transitions reflect those present in the attachment of the native ACL to bone—a highly specialized tissue transition that effectively transmits forces from soft to hard tissues. The histological examination of the implanted constructs exhibited such regions already at three months (FIG. 38F) and becoming further pronounced at six months (FIG. 38K). Interestingly, numerous interdigitated (Sharpey's) fibers were observed to project from regenerated fibrous tissue into newly generated bone tissue through similarly generated fibrocartilage (FIG. 38E,J). Thus a relatively biomimetic attachment of the silk graft to the femoral bone tunnel was achieved. In contrast, the tibial tunnel showed comparatively no fibrocartilage layer at the silk graft to bone interface (FIG. 39C,D). While we attribute this to the absence of TCP, other factors could potentially have played a role—for instance the relative mechanical stability of the different anchoring systems applied to each tunnel. In conclusion, we found that the concept of a TCP/PEEK anchored silk graft performs well as a synthetic alternative to autograft. This study provides a basis for eventual safety and efficacy testing in man.

For the animal studies an open surgical procedure (second approach) was favored to an arthroscopic approach because of a better overview and the lack of sophisticated tools for an arthroscopic anchor fixation. Because of the orientation of the femoral bone tunnel, the medial approach of accessing to the knee joint, where the patella is flipped laterally out of the way of drilling instruments, is adopted in this study. First a longitudinal median skin incision is made 5 cm proximal to the superior margin of the patella to the tibial tubercle. Then a medial parapatellar capsular approach gives the surgeon access to the knee joint. The quadriceps and patellar tendon are disconnected from the joint capsule and the vastus medialis is liberated from its insertion to the patella. Special attention should be given not to injure the patellar tendon and the medial collateral ligament. Keeping the cutting line close to the patella when liberating it from the joint capsule ensures that no damage occurs to the medial collateral ligament. Once the extensor apparatus is free, the patella can be flipped to the lateral side and the knee joint bent carefully to hold the luxated patella in its position.

For insertion of the anchor 200/insert 100 into the bore hole 2, a first tool (also denoted as insertion tool) 40 with a hollow cylindrical cross section and three protrusions (also denoted as pods) 44 was used (cf. FIG. 17). Due to the hollow cylindrical cross section, the shaft 41 of the insertion tool 40 comprises a groove 43 for receiving the flexible element 10 upon insertion of the anchor 200/insert 100 into the respective bore hole 2.

Some anchors 200 tilted in the bone tunnel 2 and once contact was lost between the instrument 40 and the anchor 200, a reinsertion of the pods 44 into corresponding recesses 202b of the head part 201 of the anchor 200 as shown in FIG. 16 was rather difficult. Hence a new insertion tool 40 with a reduced wall thickness (external radius reduced by 0.5 mm) of the distal 5 mm was developed as shown in FIG. 19. Correspondingly, the PEEK anchor 200 used with this insertion tool 40 (cf. FIG. 18) has a central opening 202 adapted to the free end 42 of the first tool 40 shown in FIG. 19.

To prevent slipping and wobbling of the drilling instrument used to drill the bore hole 2 for anchoring the device 1, which can cause an enlarged tunnel entry and a subsequent loss of fixation stability of the implant 1, a second tool 50 as shown in FIG. 20 is provided. The second tool 50 comprises a handle 51 having a free end 52, from which a cylindrical drill sleeve 53 surrounding a channel 55 for receiving a drill protrudes, wherein the drill sleeve 53 comprises a sharpened free end 54 that ensures a firm grip in the femoral notch and the handle 51 allows accurate positioning of the drilling instrument. To ensure a reproducible angulation of the femoral bone tunnel 2 the following procedures are proposed: A level rod is pressed against the anterior side of the thigh aligned with respect to the longitudinal axis of the femur; the second tool 50 (also denoted as holding instrument) is positioned at 45° angulation in the sagittal plane and 30° deviation to the lateral side. In order to ensure axial alignment of the tibia 2d and femoral bone 2 tunnels (cf. FIG. 15), a third tool 60 (for instance out of aluminum) as shown in FIG. 21 may be used. The third tool 60 comprises a first leg 61 extending along an extension direction and a second 62 and a third leg 63 connected to the free ends of the first leg 61 so that an arch is formed. A plug 64, particularly of 9 mm in diameter, particularly for form-fittedly engaging said bore hole 2, protrudes from a free end of the third leg 63 along said extension direction, wherein the second leg 62 comprises a trough-opening 65 aligned with said plug 64, in which the drill sleeve 53 of the second tool 50 can be inserted and fixed in different positions along the extension direction by a fixation means 66 such as a screw, to ensure that the second tool 50 can be adapted to different knee sizes.

After the femoral bore hole 2 is drilled (cf. FIG. 15), the plug 64 of the third tool 60 is inserted into the femoral bore hole 2, then the knee joint is extended until the drill sleeve 53 extending trough trough-opening 65 of the third tool 60 can be adjusted to the tibia edge 20d. The tibia tunnel 2d is now drilled in axial alignment with the femoral bore hole 2, shown in FIG. 15. The anchor 200 with insert 100 and silk ACL scaffold 10 is then inserted into the bore hole 2, leaving particularly the PCL just behind the ACL intact.

A preliminary study of TCP/PEEK anchored tendon autograft for CCL reconstruction in canine model was performed in healthy adult male beagles aging around one and half years old, weighing 12.0±1.1 kg (mean±SD). The ulnar carpal flexor in left forelimb was used as tendon autograft. The CCL reconstructions were performed on the right knee. The canines were thoroughly disinfected (spray) with 0.25% didecyl dimethyl ammonium bromide solution two days before operation. Antibiotics (Penicillin of 800′000 U) were given to each canine by intramuscular injection twice a day at one day before operation. A sodium pentobarbital solution of 3.5% concentration was used as anesthetic. Each canine was given 0.5 ml/kg by abdominal injection, and followed 5 minutes later with additional 0.2 ml/kg dose with vein injection. Then, the canine was positioned on its back on the operating table in a specially designed holding tray. The left forelimb and right hindleg are shaved, and washed with povidone-iodine solution thoroughly.

A tendon stripper is used to access and cut the ulnar carpal flexor from left forelimb, shown in FIG. 40A. The flexor tendon is trimmed and combined with TCP/PEEK anchor. The tendon ends are sutured with bioresorbable sutures, shown in FIG. 40B. An open surgical procedure was used as previously described and slightly adapted with the size of canine joint. First a longitudinal median skin incision was made 3 cm proximal to the superior margin of the patella to the tibia tubercle. The knee joint was accessed with medial parapatellar capsular approach. Then, the joint was bended at 90°, and native CCL was carefully cut and removed. A 5.0 mm tunnel was drilled over the footprint of ACL, with ˜15 mm in depth. To avoid damage the articular cartilage on the medial condyle, the drilling direction was 11 o'clock on the transversal plane, and 45° anterior deviation on the sagittal plane using the femoral axes as frame of the reference. A drilling sleeve was developed to prevent slipping and wobbling of the drilling instrument, which can cause an enlarged tunnel entry and a subsequent loss of fixation stability of the implant. A 5.0 mm tunnel in same axis was drilled through the tibial with a special designed synchronizing sleeve. An insertion tool for CCL graft implantation was developed, with a hollow cylindrical cross section for tendon graft, and an adapted end for holding PEEK anchor, shown in FIG. 40C. After anchoring of the TCP/PEEK scaffold into the femur tunnel, the other end of tendon graft was brought through the tibia tunnel with a specially designed retractor, shown in FIG. 40D. Then the knee joint was flexed at 30°. The tendon graft was pulled to tight, and fixed with an endobutton (PEEK, φ6 mm×2 mm, built in-house). Each canine was put into its own cage (120×100×75 cm), and unrestricted daily activities within cage is allowed. Analgesics (Pethidine of 100 mg) were given to each canine twice a day for three days right after operation to release the pain. In order to prevent infection, antibiotics (Penicillin of 800′000 U) were given to each canine twice a day until five days after operation, and spray disinfection with 0.25% didecyl dimethyl ammonium bromide solution were performed on canines as well as cages biweekly until the end of animal experiment. The normal activities and degree of lameness were monitored. This study is ongoing, although seven canines were recently euthanized at a three month time point. Preliminary CT analysis results indicate substantial formation of regenerated bone within the bone tunnel and consequent remodeling of the TCP insert (FIG. 41). Qualitatively, the tendon autograft appeared to be histologically embedded within the region of native bone/newbone/TCP, indicating a positive functional outcome. Additional biomechanical and histological analysis is also ongoing.

### REFERENCES

1. Parkkari, J., et al., The risk for a cruciate ligament injury of the knee in adolescents and young adults: a population-based cohort study of 46,500 people with a 9 year follow-up. British Journal of Sports Medicine, 2008. 42(6): p. 422-426.
2. Freeman, J. W. K., A. L, Recent Advancements in Ligament Tissue Engineering: The Usw of Various Techniques and Materials for ACL Repair. Recent Pat. Biomed. Eng, 2008. 1: p. 6.
3. Majewski, M., H. Susanne, and S. Klaus, Epidemiology of athletic knee injuries: A 10-year study. Knee, 2006. 13(3): p. 184-188.
4. Cooper, J. A., et al., Fiber-based tissue-engineered scaffold for ligament replacement: design considerations and in vitro evaluation. Biomaterials, 2005. 26(13): p. 1523-1532.
5. Vunjak-Novakovic, G., et al., Tissue engineering of ligaments. Annual Review of Biomedical Engineering, 2004. 6: p. 131-156.
6. Bach, B. R., et al., Arthroscopically assisted anterior cruciate ligament reconstruction using patellar tendon autograft—Five- to nine-year follow-up evaluation. American Journal of Sports Medicine, 1998. 26(1): p. 20-29.
7. Bach, B. R., et al., Single-incision endoscopic anterior cruciate ligament reconstruction using patellar tendon autograft—Minimum two-year follow-up evaluation. American Journal of Sports Medicine, 1998. 26(1): p. 30-40.
8. Strickland, S. M., J. D. MacGillivray, and R. F. Warren, Anterior cruciate ligament reconstruction with allograft tendons. Orthopedic Clinics of North America, 2003. 34(1): p. 41-+.
9. Badylak, S. F., et al., The Use of Xenogeneic Small-Intestinal Submucosa as a Biomaterial for Achilles-Tendon Repair in a Dog-Model. Journal of Biomedical Materials Research, 1995. 29(8): p. 977-985.
10. Milthorpe, B. K., Xenografts for Tendon and Ligament Repair. Biomaterials, 1994. 15(10): p. 745-752.
11. Maletius, W. and J. Gillquist, Long-term results of anterior cruciate ligament reconstruction with a dacron prosthesis—The frequency of osteoarthritis after seven to eleven years. American Journal of Sports Medicine, 1997. 25(3): p. 288-293.
12. Teh, T. K. H., S. L. Toh, and J. C. H. Goh, Aligned Hybrid Silk Scaffold for Enhanced Differentiation of Mesenchymal Stem Cells into Ligament Fibroblasts. Tissue Engineering Part C-Methods, 2011. 17(6): p. 687-703.
13. Miller, S. L. and J. N. Gladstone, Graft selection in anterior cruciate ligament reconstruction. Orthopedic Clinics of North America, 2002. 33(4): p. 675-+.
14. Liu, H. F., et al., A comparison of rabbit mesenchymal stem cells and anterior cruciate ligament fibroblasts responses on combined silk scaffolds. Biomaterials, 2008. 29(10): p. 1443-1453.
15. Shen, W. L., et al., The effect of incorporation of exogenous stromal cell-derived factor-1 alpha within a knitted silk-collagen sponge scaffold on tendon regeneration. Biomaterials, 2010. 31(28): p. 7239-7249.
16. Sahoo, S., S. L. Toh, and J. C. H. Goh, A bFGF-releasing silk/PLGA-based biohybrid scaffold for ligament/tendon tissue engineering using mesenchymal progenitor cells. Biomaterials, 2010. 31(11): p. 2990-2998.
17. Fan, H. B., et al., Anterior cruciate ligament regeneration using mesenchymal stem cells and silk scaffold in large animal model. Biomaterials, 2009. 30(28): p. 4967-4977.
18. Ge, Z. G., et al., Biomaterials and scaffolds for ligament tissue engineering. Journal of Biomedical Materials Research Part A, 2006. 77A(3): p. 639-652.
19. D. I. Zeugolis, J. C. Y. C., and A. Pandit, Tendons: Engineering of Functional Tissues. Tissue Engineering, 2011: p. 36.
20. Teh, T. K. H., S. L. Toh, and J. C. H. Goh, Optimization of the silk scaffold sericin removal process for retention of silk fibroin protein structure and mechanical properties. Biomedical Materials, 2010. 5(3).
21. Wang, X., et al., Improved human tenocyte proliferation and differentiation in vitro by optimized silk degumming. Biomedical Materials, 2011. 6(3).
22. Moy, R. L., A. Lee, and A. Zalka, Commonly Used Suture Materials in Skin Surgery. American Family Physician, 1991. 44(6): p. 2123-2128.
23. Altman, G. H., et al., Silk-based biomaterials. Biomaterials, 2003. 24(3): p. 401-416.
24. Wang, Y. Z., et al., Stem cell-based tissue engineering with silk biomaterials. Biomaterials, 2006. 27(36): p. 6064-6082.
25. Minoura, N., et al., Attachment and Growth of Cultured Fibroblast Cells on Silk Protein Matrices. Journal of Biomedical Materials Research, 1995. 29(10): p. 1215-1221.
26. Inouye, K., et al., Use of Bombyx mori silk fibroin as a substratum for cultivation of animal cells. Journal of Biochemical and Biophysical Methods, 1998. 37(3): p. 159-164.
27. Zhang, Q. A., S. Q. Yan, and M. Z. Li, Porous Materials Based on Bombyx mori Silk Fibroin. Textile Bioengineering and Informatics Symposium Proceedings, Vols 1-3, 2010: p. 254-261.
28. Sandmann, G. H. and T. Tischer, Tissue Engineering of the ACL—Efforts and Achievements. Anterior Cruciate Ligament (Ad): Causes of Injury, Adverse Effects and Treatment Options, 2010: p. 225-246.
29. Panas, E., C. J. Gatt, and M. G. Dunn, In Vitro Analysis of a Tissue-Engineered Anterior Cruciate Ligament Scaffold. 2009 35th Annual Northeast Bioengineering Conference, 2009: p. 286-287.
30. Laurencin, C. T. and J. W. Freeman, Ligament tissue engineering: An evolutionary materials science approach. Biomaterials, 2005. 26(36): p. 7530-7536.
31. Weitzel, P. P., et al., Future direction of the treatment of ACL ruptures. Orthopedic Clinics of North America, 2002. 33(4): p. 653-+.
32. Horan, R. L., et al., Yarn design for functional tissue engineering. Journal of Biomechanics, 2006. 39(12): p. 2232-2240.
33. Altman, G. H., et al., Silk matrix for tissue engineered anterior cruciate ligaments. Biomaterials, 2002. 23(20): p. 4131-4141.
34. Min, B. M., et al., Formation of silk fibroin matrices with different texture and its cellular response to normal human keratinocytes. International Journal of Biological Macromolecules, 2004. 34(5): p. 281-288.
35. Min, B. M., et al., Electrospinning of silk fibroin nanofibers and its effect on the adhesion and spreading of normal human keratinocytes and fibroblasts in vitro. Biomaterials, 2004. 25(7-8): p. 1289-1297.
36. Fang, Q., et al., In vitro and in vivo research on using Antheraea pernyi silk fibroin as tissue engineering tendon scaffolds. Materials Science & Engineering C-Biomimetic and Supramolecular Systems, 2009. 29(5): p. 1527-1534.
37. Chen, X., et al., Synergic Combination of Collagen Matrix with Knitted Silk Scaffold Regenerated Ligament with More Native Microstructure in Rabbit Model. 13th International Conference on Biomedical Engineering, Vols 1-3, 2009. 23(1-3): p. 1195-1198.
38. Altman, G. H., et al., “The use of long-term bioresorbable scaffolds for anterior cruciate ligament repair” (vol 16, pg 177, 2008). Journal of the American Academy of Orthopaedic Surgeons, 2008. 16(8): p. 22a-22a.
39. Horan, R. L., SeriACL™ Device (Gen IB) Trial for Anterior Cruciate Ligament (ACL) Repair, Serica Technologies, Inc. 2009(NCT00775892).
40. Bernardino, S., ACL prosthesis: any promise for the future? (Retracted Article. See vol 18, pg 1814, 2010). Knee Surgery Sports Traumatology Arthroscopy, 2010. 18(6): p. 797-804.
41. Mascarenhas, R. and P. B. MacDonald, Anterior cruciate ligament reconstruction: a look at prosthetics—past, present and possible future. Mcgill J Med, 2008. 11(1): p. 29-37.
42. Wen, C. Y., et al., The Use of Brushite Calcium Phosphate Cement for Enhancement of Bone-Tendon Integration in an Anterior Cruciate Ligament Reconstruction Rabbit Model. Journal of Biomedical Materials Research Part B—Applied Biomaterials, 2009. 89B(2): p. 466-474.
43. Huangfu, X. Q. and J. Z. Zhao, Tendon-bone healing enhancement using injectable tricalcium phosphate in a dog anterior cruciate ligament reconstruction model. Arthroscopy—the Journal of Arthroscopic and Related Surgery, 2007. 23(5): p. 455-462.
44. Soon, M. Y. H., et al., An analysis of soft tissue allograft anterior cruciate ligament reconstruction in a rabbit model—A short-term study of the use of mesenchymal stem cells to enhance tendon osteointegration. American Journal of Sports Medicine, 2007. 35(6): p. 962-971.
45. Lim, J. K., et al., Enhancement of tendon graft osteointegration using mesenchymal stem cells in a rabbit model of anterior cruciate ligament reconstruction. Arthroscopy—the Journal of Arthroscopic and Related Surgery, 2004. 20(9): p. 899-910.
46. Rodeo, S. A., et al., Use of recombinant human bone morphogenetic protein-2 to enhance tendon healing in a bone tunnel. American Journal of Sports Medicine, 1999. 27(4): p. 476-488.
47. Yu, Y., et al., Bone morphogenetic proteins and Smad expression in ovine tendon-bone healing. Arthroscopy—the Journal of Arthroscopic and Related Surgery, 2007. 23(2): p. 205-210.
48. Beynnon, B. D. and A. A. Amis, In vitro testing protocols for the cruciate ligaments and ligament reconstructions. Knee Surg Sports Traumatol Arthrosc, 1998: p. 7.
49. Nurmi, J., P. Kannus, and S. H, Interference Screw Fixation of Soft Tissue Grafts in Anterior Cruciate Ligament Reconstruction: Part 2. Am J Sports Med, 2004. 32(2): p. 5.
50. Coleridge, S. D. and A. A. Amis, A comparison of five tibial-fixation systems in hamstring-graft anterior cruciate ligament reconstruction. Knee Surgery Sports Traumatology Arthroscopy, 2004. 12(5): p. 391-397.
51. Woo, S. L. Y., et al., Tensile Properties of the Human Femur-Anterior Cruciate Ligament-Tibia Complex—the Effects of Specimen Age and Orientation. American Journal of Sports Medicine, 1991. 19(3): p. 217-225.
52. Noyes, F. R. and E. S. Grood, Strength of Anterior Cruciate Ligament in Humans and Rhesus-Monkeys. Journal of Bone and Joint Surgery-American Volume, 1976. 58(8): p. 1074-1082.

1. Device for fixing a flexible element (10), particularly in the form of a natural or synthetical ligament or tendon, to a bone (20), comprising:
an insert (100) being designed to hold said flexible element (10), and
an anchor (200), wherein the insert (100) is designed to be inserted into said anchor (200), and wherein the anchor element (200) is designed to be inserted into a bore hole (2) of said bone (20) together with said insert (100) inserted into the anchor (200) to fix the flexible element (10) to the bone (20).

2. Device as claimed in claim 1, characterized in that the insert (100) is formed out of an osteoinductive and/or osteoconductive material or comprises an osteoinductive and/or osteoconductive material.

3. Device as claimed in claim 1, characterized in that the anchor (200) is designed to be inserted into said bore hole (2) along an insertion direction (Z) together with said insert (100) inserted into the anchor (200).

4. Device as claimed in claim 1, characterized in that the anchor (200) comprises a head part (201) and a first and a second leg (210, 220) protruding from said head part (201), wherein particularly the legs (210, 220) are integrally formed with the head part (201) and wherein particularly the legs (210, 220) protrude from the head part (201) in the insertion direction (Z).

5. Device as claimed in claim 4, characterized in that the head part (201) comprises an annular shape, wherein particularly the head part (201) comprises an opening (202) for passing through said flexible element (10).

6. Device as claimed in claim 4, characterized in that the head part (201) comprises two opposing cut-outs (203, 204) for bypassing the flexible element (10), wherein each cut-out (203, 204) is formed in a boundary region of the head part (201).

7. Device as claimed in claim 4, characterized in that the insert (100) is arranged between the legs (210, 220) of the anchor (200) when the insert (100) is inserted into the anchor (200).

8. Device as claimed in claim 4, characterized in that the insert (100) comprises a first and a second guiding recess (110, 120) being designed to receive the legs (210, 220) in a form fitting manner when the insert (100) is inserted into the anchor (200).

9. Device as claimed in claim 8, characterized in that each guiding recess (110, 120) is delimited by a surface (110a, 120a) of the insert (100), wherein the two surfaces (110a, 120a) face away from each other, and two opposing boundary regions (112, 113, 122, 123) protruding from the respective surface (110a, 120a), wherein particularly the two surfaces (110a, 120a) are convex.

10. Device as claimed in claim 9, characterized in that each boundary region (112, 113, 122, 123) comprises a contact surface (112a, 113a, 122a, 123a) being designed to contact the bone (20) when the anchor (200) is inserted into the bore hole (2) of the bone (20) together with the insert (100) as intended, which contact surface (112a, 113a, 122a, 123a) extends along the respective guiding recess (110, 120).

11. Device as claimed in claim 1, characterized in that the anchor (200) comprises an outside (200a) for contacting the bone (20), wherein particularly said outside (200a) comprises a toothed surface, and wherein particularly the respective contact surface (112a, 113a, 122a, 123a) is flush with said outside (200a) of the anchor (200) when the insert (100) is inserted into the anchor (200) as intended.

12. Device as claimed in claim 4, characterized in that a region (110a, 120a) of the insert (100) is tapered so that upon inserting the insert (100) into the anchor (200), the insert (100), particularly by means of the surfaces (110a, 120a) of the insert (100), presses the legs (210, 220) away from each other, wherein particularly the anchor (200) is designed to be inserted into the bore hole (2) in the insertion direction (Z) with the insert (100) being inserted into the anchor (200) in a first position, in which the insert (100) is not fully inserted into the anchor (200), wherein particularly the insert (100) is designed to be pulled into a second position counter to the insertion direction (Z) when the anchor (200) is inserted into the bore hole (2) of the bone (20) as intended, in which second position the insert (100) is fully inserted into the anchor (200) and presses the legs (210, 220) against the bone (20).

13. Device as claimed in claim 4, characterized in that each of the legs (210, 220) comprises an inner surface (210a, 220a), wherein the two inner surfaces (210a, 220a) face each other, and wherein particularly said inner surfaces (210a, 220a) are concave.

14. Device as claimed in claim 9, characterized in that each inner surface (210a, 220a) is designed to rest on an associated surface (110a, 120a) of a guiding recess (110, 120).

15. Device as claimed in claim 1, characterized in that the insert (100) comprises a first wall region (101) and a second wall region (102), wherein particularly the first guiding recess (110) is formed in the first wall region (101), and wherein particularly the second guiding recess (120) is formed in the second wall region (102).

16. Device as claimed in claim 15, characterized in that the two wall regions (101, 102) are integrally connected by a connecting region (103), wherein particularly the connecting region (103) comprises a surface (103a) for contacting the flexible element (10), wherein particularly said surface (103a) is concave.

17. Device as claimed in claim 1, characterized in that the insert (100) comprises a groove (104) for receiving the flexible element (10), wherein particularly said groove (104) is delimited by the two wall regions (101, 102) and the connecting region (103).

18. Device as claimed in claim 1, characterized in that the device (1) comprises said flexible element (10).

19. Device as claimed in claim 18, characterized in that the flexible element (10) is laid around the insert (100), particularly around the connecting region (103), particularly such that it contacts the insert (100), wherein particularly said flexible element (10) is arranged in said groove (104).

20. Device as claimed in claim 5, characterized in that the flexible element (10) passes through the opening (202) of the head part (201).

21. Device as claimed in claim 6, characterized in that the flexible element (10) passes by the cut-outs (203, 204) of the head part (201).

22. Device as claimed in claim 18, characterized in that the flexible element (10) is a natural ligament or tendon.

23. Device as claimed in claim 18, characterized in that the flexible element (10) is a synthetic ligament or tendon, particularly an ACL scaffold.

24. Device as claimed in claim 18, characterized in that the flexible element (10) comprises two twisted cords (300), each cord (300) comprising 144 twisted yarns (301), each yarn (301) comprising two twisted bundles (302), each bundle comprising 6 fibres (303), which fibres (303) particularly comprise fibroin.

25. Device as claimed in claim 18, characterized in that the flexible element (10) comprises three braided cords (300), each cord (300) comprising 96 twisted yarns (301), each yarn (301) comprising two twisted bundles (302), each bundle (302) comprising 6 fibres (303), which fibres (303) particularly comprise fibroin.

26. Device as claimed in claim 1, characterized in that said insert (100) comprises one of the following substances: tricalcium phosphate, hydroxylapatite, calcium phosphate, calcium silicate, or silicate-substituted calcium phosphate.

27. Device as claimed in claim 1, characterized in that the anchor (200) comprises one of the following substances: polyether ether ketone, poly lactic acid, poly(lactic-co-glycolic acid), poly-ε-caprolactone, a titanium-based alloy, or a magnesium-based alloy.

28. Tool set for inserting a device (1) according to claim 1 into a bore hole (2) in a bone (20), comprising at least a first tool (40) for pressing the device (1) into said bore hole (2), wherein said first tool (40) comprises an elongated shaft (41) having a free end (42) that is designed to engage with the anchor (200), particularly with the head part (201) of the anchor (200), for pressing the device (1) into said bore hole (2), wherein said elongated shaft (41) comprises a groove (43) for receiving the flexible element (10) upon insertion of the device (1) into the bore hole (2) of the bone (20).
